(19) United States
(12) Patent Application Publication
(10) Pub. No.: US 2007/0207175 A1
(43) Pub. Date: Sep. 6, 2007

(54) Biaromatic compound activators of PPARgamma receptors and cosmetic/pharmaceutical compositions comprised thereof

(75) Inventors: Laurence Clary, La Colle Sur Loup (FR); Jean-Guy Boiteau, Saint Aunes (FR); Corinne Millois Barbuis, Saint Raphael (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 11/707,125

(22) Filed: Feb. 16, 2007

(30) Foreign Application Priority Data
Aug. 17, 2004 (FR) 04/08932
Aug. 12, 2005 (EP) PCT/EP05/09989

(51) Int. Cl. A61K 31/495; A61K 31/40; A61K 31/195; C07C 237/40

(52) U.S. Cl. 424/401; 514/563; 514/255.01; 514/408; 514/489; 548/561; 544/386; 560/24; 562/450

(57) ABSTRACT

Novel biaromatic compounds having the general formula (I): 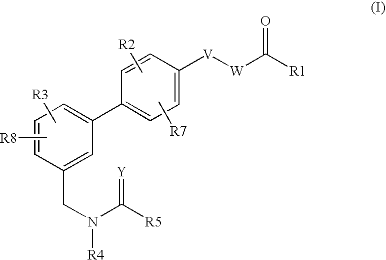 and cosmetic/pharmaceutical compositions comprised thereof are useful in human or veterinary medicine (in dermatology and also in the fields of cardiovascular diseases, of immune diseases and/or of diseases related to the metabolism of lipids) or, alternatively, in cosmetic compositions.

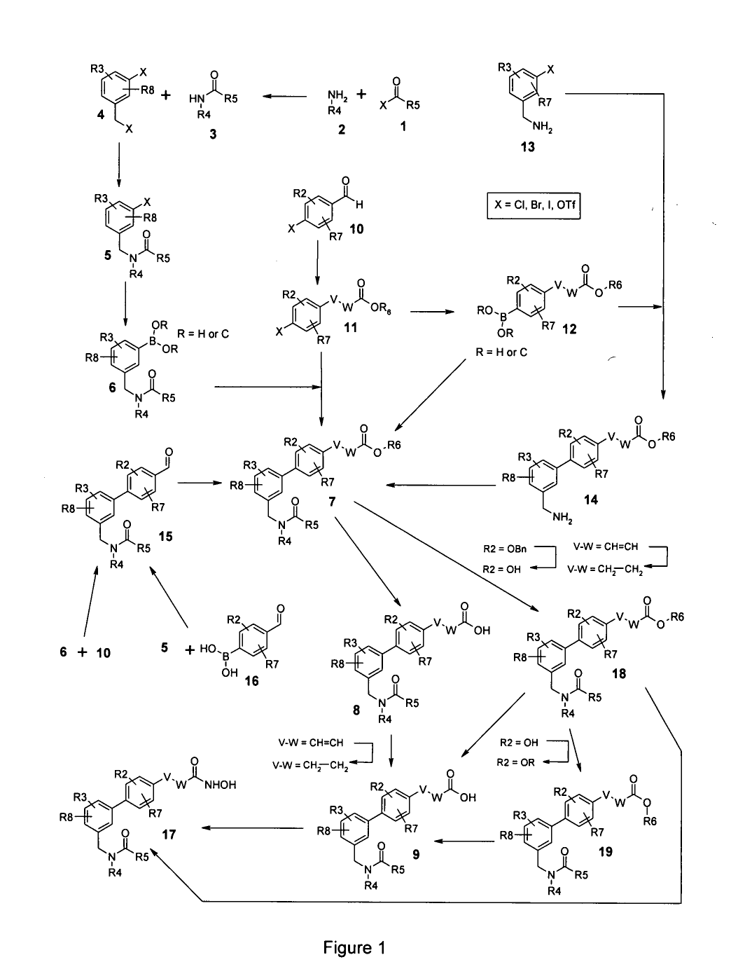

### CROSS-REFERENCE TO PRIORITY/PCT/PROVISIONAL APPLICATIONS

This application claims priority under 35 U.S.C. § 119 of FR 04/08932, filed Aug. 17, 2004, and of Provisional Application No. 60/607,782, filed Sep. 8, 2004, and is a continuation of PCT/EP 2005/009989 filed Aug. 12, 2005 and designating the United States, published in the English language as WO 2006/018325 A1 on Feb. 23, 2006, each hereby expressly incorporated by reference in its entirety and each assigned to the assignee hereof.

### CROSS-REFERENCE TO COMPANION APPLICATION

Copending application Ser. No. ______ [Attorney Docket No. 1034227-000835] filed concurrently herewith, hereby also expressly incorporated by reference and also assigned to the assignee hereof.

### BACKGROUND OF THE INVENTION

1. Technical Field of the Invention

The present invention relates, as novel and useful industrial products, to a novel class of biaromatic compounds which are activators of receptors of Peroxisome Proliferator-Activated Receptor type of subtype γ (PPARγ). This invention also relates to a process for the preparation of same and to their formulation into pharmaceutical compositions useful in human or veterinary medicine or, alternatively, into cosmetic compositions.

2. Description of Background and/or Related and/or Prior Art

The activity of receptors of PPAR type has been the subject of many studies. See, for example, the publication entitled “Differential Expression of Peroxisome Proliferator-Activated Receptor Subtypes During the Differentiation of Human Keratinocytes”, Michel Rivier et al., J. Invest. Dermatol., 111, 1998, pp 1116-1121, in which a large number of bibliographic references relating to receptors of PPAR type are listed. See also the report entitled “The PPARs: From Orphan Receptors to Drug Discovery”, Timothy M. Willson, Peter J. Brown, Daniel D. Sternbach and Brad R. Henke, J. Med. Chem., 2000, Vol. 43, pp 527-550.

PPAR receptors activate transcription by binding to elements of DNA sequences, known as peroxisome proliferator response elements (PPRE), in the form of a heterodimer with retinoid X receptors (known as RXRs).

Three subtypes of human PPARs have been identified and described: PPARα, PPARγ and PPARδ (or NUC1).

PPARα is mainly expressed in the liver, while PPARδ is ubiquitous.

PPARγ is the most widely studied of the three subtypes. All the references suggest a critical role for PPARγs in the regulation of the differentiation of adipocytes, where it is strongly expressed. It also plays a key role in systemic lipid homeostasis.

It has been described, in particular, in WO 96/33724 that compounds which are selective for PPARγs, such as a prostaglandin J₂ or D₂, are potential active principles in the treatment of obesity and diabetes.

Furthermore, in WO 02/12210 and WO 03/055867 the assignee hereof describes the formulation of biaromatic compounds which are activators of receptors of PPARγ type into a pharmaceutical composition, the composition being useful for the treatment of skin disorders related to an anomaly in the differentiation of the epidermal cells.

Need continues to exist for novel compounds exhibiting a good activity and advantageous pharmaceutical properties.

### SUMMARY OF THE INVENTION

A novel family of compounds has now been developed exhibiting the advantage of being 10 to 100 times more active than the compounds identified in the preceding WO 02/12210 and WO 03/055867 with regard to PPARγ receptors. Furthermore, in addition to their better activity, certain of the compounds according to the present invention are obtained in the solid form. The synthesis thereof and the purification thereof thus are easier.

Too, the use of solid compounds makes it possible to avoid the disadvantages exhibited by oils in the context of the pharmaceutical development due to the residual solvents which may be present therein.

Thus, the present invention features biaromatic compounds having the following general formula (I): 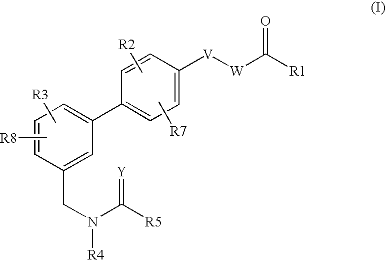
in which:

R1 is a hydroxyl radical, an —OR6 radical or a hydroxylamine radical; wherein R6 is as defined below,

R2 and R3, which may be identical or different, are each a hydrogen atom, a halogen atom, an alkyl radical having from 1 to 12 carbon atoms, a hydroxyl radical, an alkoxy radical, a polyether radical, an aralkyl radical, an aryl radical or an amino radical which can be substituted by one or two identical or different radicals selected from among an alkyl radical having from 1 to 12 carbon atoms or an aralkyl radical;

R4 is a hydrogen atom or a lower alkyl radical having from 1 to 4 carbon atoms;

R5 is an alkyl radical having from 1 to 12 carbon atoms, an aryl radical, an aralkyl radical, a heteroaryl radical, a heterocyclic radical or a 9-fluorenylmethyl radical;

R6 is an alkyl, aryl or aralkyl radical;

R7 and R8, which may be identical or different, are each a hydrogen atom, a halogen atom, an alkyl radical having from 1 to 12 carbon atoms, a hydroxyl radical, an alkoxy radical, a polyether radical, an aralkyl radical, an aryl radical or an amino radical which can be substituted by one or two identical or different radicals selected from among an alkyl radical having from 1 to 12 carbon atoms or an aralkyl radical;

Y is an oxygen or sulfur atom;

V—W is a carbon-carbon single or double bond, namely, a CH₂—CH₂ or CH═CH sequence,

and the salts thereof.

### BRIEF DESCRIPTION OF THE DRAWINGS

The FIGURE of Drawing shows a variety of reaction schemes for the ultimate preparation of the biaromatic compounds according to the present invention.

### DETAILED DESCRIPTION OF BEST MODE AND SPECIFIC/PREFERRED EMBODIMENTS OF THE INVENTION

In particular, when the compounds according to the invention are provided in the form of salts, they are salts of an alkali metal, in particular a sodium or potassium salt, or of an alkaline earth metal or salts of organic amines, more particularly of amino acids, such as arginine or lysine.

When the compounds according to the invention have an amine functional group and are provided in the form of salts of this amine, they are salts of an inorganic acid, such as, for example, hydrochloric acid, sulfuric acid or hydrobromic acid, or salts of an organic acid, such as, for example, acetic acid, triflic acid, tartaric acid, oxalic acid, citric acid or nitric acid.

According to the present invention, the term “hydroxyl radical” means the —OH radical.

According to the present invention, the term “alkyl radical having from 1 to 12 carbon atoms” means a linear, branched or cyclic and saturated or unsaturated carbon chain which can be interrupted by a heteroatom and which can be substituted by one or more radicals selected from among a halogen atom, a hydroxyl radical, an alkoxy radical or a heterocyclic radical and the alkyl radicals are preferably the methyl, ethyl, propyl, isopropyl, butyl, tert-butyl, isoamyl, amyl, hexyl, heptyl, octyl, decyl, cyclohexyl, methylcyclohexyl, methylcyclobutyl, methylcyclopentyl or methylcyclohexyl radicals.

The term “lower alkyl radical having from 1 to 4 carbon atoms” will preferably mean a methyl, ethyl, propyl, methylcyclopropyl, isopropyl, tert-butyl or n-butyl radical.

The term “aryl radical” means a phenyl, biphenyl, cinnamyl or naphthyl radical which can be substituted by a halogen atom, a CF₃ radical, an alkyl radical, an alkoxy radical, a nitro functional group, a polyether radical, an aryl radical, a benzoyl radical, an alkyl ester group, a carboxylic acid, a hydroxyl radical optionally protected by an acetyl or benzoyl group or an amino radical optionally protected by an acetyl or benzoyl group or optionally substituted by at least one alkyl having from 1 to 12 carbon atoms, an aralkoxy radical, a phenoxy radical or an amide radical H₂NCO.

The term “aralkyl radical” means an alkyl radical having from 1 to 12 carbon atoms which is substituted by an aryl radical or by a heteroaryl radical.

The term “halogen atom” means a fluorine, chlorine, bromine or iodine atom.

The term “alkoxy radical” means an oxygen atom substituted by an alkyl radical having from 1 to 12 carbon atoms and the alkoxy radicals are preferably the methoxy, ethoxy, isopropyloxy, n-propyloxy, tert-butoxy, n-butoxy, n-pentyloxy, n-hexyloxy, cyclopropylmethoxy, cyclobutylmethoxy, cyclopentylmethoxy or cyclohexylmethoxy radicals.

The term “aralkoxy radical” means an oxygen atom substituted by an aralkyl radical.

The term “polyether radical” means a radical having from 1 to 7 carbon atoms which is interrupted by at least one oxygen atom and preferably the radicals such as methoxyethoxy, ethoxyethoxy or methoxyethoxyethoxy radicals.

The term “heteroaryl radical” means an aryl radical which is interrupted by one or more heteroatoms, such as the pyridyl, furyl, thienyl, isoxazolyl, oxadiazolyl, oxazolyl, isothiazolyl, quinozalinyl, benzothiadiazolyl, benzimidazolyl, indolyl or benzofuranyl radical, and which is optionally substituted by at least one halogen, one alkyl radical having from 1 to 12 carbon atoms, one alkoxy radical, one aryl radical, one nitro functional group, one polyether radical, one heteroaryl radical, one benzoyl radical, one alkyl ester group, one carboxylic acid, one hydroxyl optionally protected by an acetyl or benzoyl group or one amino radical optionally protected by an acetyl or benzoyl group or optionally substituted by at least one alkyl having from 1 to 12 carbon atoms.

The term “heterocyclic radical” means preferably a morpholino, pyrrolidino, piperidino, piperazino, 2-oxopiperidin-1-yl and 2-oxopyrrolidin-1-yl radical which are optionally substituted by at least one alkyl radical having from 1 to 12 carbon atoms, one alkoxy radical, one aryl radical, one nitro functional group, one polyether radical, one heteroaryl radical, one benzoyl radical, one alkyl ester group, one carboxylic acid, one hydroxyl optionally protected by an acetyl or benzoyl group or one amino radical optionally protected by an acetyl or benzoyl group or optionally substituted by at least one alkyl having from 1 to 12 carbon atoms.

The term “hydroxylamine radical” means the —NHOH sequence.

The term “alkyl ester radical” means a carboxylate functional group substituted by an alkyl radical having 1 to 6 carbon atoms.

Exemplary compounds of formula (I) according to the present invention, whether alone or as a mixture, include:
1. 3-{3′-[((Methyl)(octanoyl)amino)methyl]biphenyl-4-yl}acrylic acid,
2. 3-{3′-[((Methyl)(octanoyl)amino)methyl]biphenyl-4-yl}propanoic acid,
3. 3-{3-Fluoro-3′-[((methyl)(octanoyl)amino)methyl]biphenyl-4-yl}acrylic acid,
4. 3-{3-Fluoro-3′-[((methyl)(octanoyl)amino)methyl]biphenyl-4-yl}propanoic acid,
5. 3-[3′-((Octanoylamino)methyl)biphenyl-4-yl]acrylic acid,
6. 3-[3′-((Octanoylamino)methyl)biphenyl-4-yl]propanoic acid,
7. 3-{3-Hydroxy-3′-[((methyl)(octanoyl)amino)methyl]biphenyl-4-yl}propanoic acid,
8. 3-{2-Butoxy-3′-[((methyl)(octanoyl)amino)methyl]biphenyl-4-yl}propanoic acid,
9. 3-{2-Butoxy-3′-[((hexanoyl)(methyl)amino)methyl]biphenyl-4-yl}propanoic acid,
10. 3-{2-Butoxy-3′-[((methyl)(pentanoyl)amino)methyl]biphenyl-4-yl}propanoic acid,
11. 3-{2-Butoxy-3′-[((heptanoyl)(methyl)amino)methyl]biphenyl-4-yl}propanoic acid,
12. 3-(2-Butoxy-3′-{[(4-ethoxybenzoyl)(methyl)amino]-methyl}biphenyl-4-yl)propanoic acid,
13. 3-(2-Butoxy-3′-{[(4-butoxybenzoyl)(methyl)amino]-methyl}biphenyl-4-yl)propanoic acid,
14. 3-{3′-[((Benzoyl)(methyl)amino)methyl]-2-butoxy-biphenyl-4-yl}propanoic acid,
15. 3-(2-Butoxy-3′-{[(4-methoxybenzoyl)(methyl)amino]-methyl}biphenyl-4-yl)propanoic acid,
16. 3-(2-Butoxy-3′-{[(3-methoxybenzoyl)(methyl)amino]-methyl}biphenyl-4-yl)propanoic acid,
17. (E)-3-{2-Fluoro-3′-[((methyl)(octanoyl)amino)methyl]biphenyl-4-yl}acrylic acid,
18. 3-{2-Fluoro-3′-[((methyl)(octanoyl)amino)methyl]biphenyl-4-yl}propanoic acid,
19. 3-{2-(2-Methoxyethoxy)-3′-[((methyl)(octanoyl)amino)methyl]biphenyl-4-yl}propanoic acid,
20. 3-{2-(3-Methylbutoxy)-3′-[((methyl)(octanoyl)amino)methyl]biphenyl-4-yl}propanoic acid,
21. 3-{2-(3-Chloropropoxy)-3′-[((methyl)(octanoyl)amino)methyl]biphenyl-4-yl}propanoic acid,
22. 3-{2-Methoxy-3′-[((methyl)(octanoyl)amino)methyl]biphenyl-4-yl}propanoic acid,
23. 3-{2-Methyl-3′-[((methyl)(octanoyl)amino)methyl]biphenyl-4-yl}propanoic acid,
24. 3-(3′-{[Methyl(4-phenylbutyryl)amino]methyl}-biphenyl-4-yl)propanoic acid,
25. (E)-3-{2′-Methyl-5′-[((methyl)(octanoyl)amino)methyl]biphenyl-4-yl}acrylic acid,
26. 3-{2′-Methyl-5′-[((methyl)(octanoyl)amino)methyl]biphenyl-4-yl}propanoic acid,
27. (E)-3-{2-Benzyloxy-3′-[((methyl)(octanoyl)amino)methyl]biphenyl-4-yl}acrylic acid,
28. 3-{2-Benzyloxy-3′-[((methyl)(octanoyl)amino)methyl]biphenyl-4-yl}propanoic acid,
29. 3-{3′-[((Methyl)(octanoyl)amino)methyl]-2-propoxy-biphenyl-4-yl}propanoic acid,
30. (E)-3-{3,5-Difluoro-3′-[((methyl)(octanoyl)amino)methyl]biphenyl-4-yl}acrylic acid,
31. 3-{3,5-Difluoro-3′-[((methyl)(octanoyl)amino)methyl]biphenyl-4-yl}propanoic acid,
32. 3-(3′-{[(4-Butoxybenzoyl)(methyl)amino]methyl}-biphenyl-4-yl)propanoic acid,
33. 3-(3′-{[(4-Butoxybenzoyl)(ethyl)amino]methyl}-biphenyl-4-yl)propanoic acid,
34. 3-{2-Cyclopropylmethoxy-3′-[((methyl)(octanoyl)amino)methyl]biphenyl-4-yl}propanoic acid,
35. 3-{2-Ethoxy-3′-[((methyl)(octanoyl)amino)methyl]biphenyl-4-yl}propanoic acid,
36. 3-[3′-[((Methyl)(octanoyl)amino)methyl]-2-(3,3,3-trifluoropropoxy)biphenyl-4-yl]propanoic acid,
37. 3-[3′-[((Methyl)(octanoyl)amino)methyl]-2-(4,4,4-trifluorobutoxy)biphenyl-4-yl]propanoic acid,
38. 3-{2-(3-Hydroxypropoxy)-3′-[((methyl)(octanoyl)amino)methyl]biphenyl-4-yl}propanoic acid,
39. 3-{2-(4-Hydroxybutoxy)-3′-[((methyl)(octanoyl)amino)methyl]biphenyl-4-yl}propanoic acid,
40. 3-{2-(3-Fluorobenzyloxy)-3′-[((methyl)(octanoyl)amino)methyl]biphenyl-4-yl}propanoic acid,
41. 3-{2-(4-Fluorobenzyloxy)-3′-[((methyl)(octanoyl)amino)methyl]biphenyl-4-yl}propanoic acid,
42. 3-{3′-[((Methyl)(octanoyl)amino)methyl]-2-(pentyloxy)biphenyl-4-yl}propanoic acid,
43. 3-{3′-[((Hexanoyl)(methyl)amino)methyl]-2-(pentyloxy)biphenyl-4-yl}propanoic acid,
44. 3-{2-(2-Ethoxyethoxy)-3′-[((hexanoyl)(methyl)amino)methyl]biphenyl-4-yl}propanoic acid,
45. 3-{2-(2-(Diethylamino)ethoxy)-3′-[((methyl)(octanoyl)amino)methyl]biphenyl-4-yl}propanoic acid,
46. 3-[3′-[((Methyl)(octanoyl)amino)methyl]-2-(2-(morpholin-4-yl)ethoxy)biphenyl-4-yl]propanoic acid,
47. 3-{2-Amino-3′-[((hexanoyl)(methyl)amino)methyl]biphenyl-4-yl}propanoic acid,
48. 3-{2-Butylamino-3′-[((hexanoyl)(methyl)amino)methyl]biphenyl-4-yl}propanoic acid,
49. 3-{2-Benzylamino-3′-[((hexanoyl)(methyl)amino)methyl]biphenyl-4-yl}propanoic acid,
50. 3-{2-Diethylamino-3′-[((hexanoyl)(methyl)amino)methyl]biphenyl-4-yl}propanoic acid,
51. 3-{3′-[((Hexanoyl)(methyl)amino)methyl]-2-(propylamino)biphenyl-4-yl}propanoic acid,
52. 3-{2-(4-Fluorobenzylamino)-3′-[((hexanoyl)(methyl)amino)methyl]biphenyl-4-yl}propanoic acid,
53. 3-{2-Butylamino-3′-[((methyl)(octanoyl)amino)methyl]biphenyl-4-yl}propanoic acid,
54. 3-{2-Benzylamino-3′-[((methyl)(octanoyl)amino)methyl]biphenyl-4-yl}propanoic acid,
55. 3-{2-Diethylamino-3′-[((methyl)(octanoyl)amino)methyl]biphenyl-4-yl}propanoic acid,
56. 3-{3′-[((Methyl)(octanoyl)amino)methyl]-2-(propylamino)biphenyl-4-yl}propanoic acid,
57. 3-{2-(4-Fluorobenzylamino)-3′-[((methyl)(octanoyl)amino)methyl]biphenyl-4-yl}propanoic acid,
58. 3-{2-Cyclohexylmethoxy-3′-[((methyl)(octanoyl)amino)methyl]biphenyl-4-yl}propanoic acid,
59. 3-{2-Cyclopentylmethoxy-3′-[((methyl)(octanoyl)amino)methyl]biphenyl-4-yl}propanoic acid,
60. N-[2′-(2-Cyclobutylethoxy)-4′-(2-(hydroxycarbamoyl)ethyl)biphenyl-3-ylmethyl](methyl)octanamide,
61. 3-[3′-[((Methyl)(octanoyl)amino)methyl]-2-(3-(trifluoromethyl)benzyloxy)biphenyl-4-yl]propanoic acid,
62. 3-[3′-[((Hexanoyl)(methyl)amino)methyl]-2-(4-(trifluoromethyl)benzyloxy)biphenyl-4-yl]propanoic acid,
63. 3-{2-(3-Carbamoylbenzyloxy)-3′-[((methyl)(octanoyl)amino)methyl]biphenyl-4-yl}propanoic acid,
64. 3-[3′-[((Methyl)(octanoyl)amino)methyl]-2-(2-(piperazin-1-yl)ethoxy)biphenyl-4-yl]propanoic acid,
65. 3-[3′-[((Methyl)(octanoyl)amino)methyl]-2-(2-(pyrrolidin-1-yl)ethoxy)biphenyl-4-yl]propanoic acid,
66. 3-[2-(3-Methoxybenzyloxy)-3′-({[3-(3-methoxyphenyl)propionyl](methyl)amino}methyl)biphenyl-4-yl]propanoic acid,
67. 3-[2-(4-(tert-Butyl)benzyloxy)-3′-({methyl[3-(3-phenoxyphenyl)propionyl]amino}methyl)biphenyl-4-yl]propanoic acid,
68. 3-(2-(3,5-Dimethoxybenzyloxy)-3′-{[methyl(3-phenoxybenzoyl)amino]methyl}biphenyl-4-yl)propanoic acid,
69. 3-[3′-{[Methyl(4-phenoxybenzoyl)amino]methyl}-2-(3-(trifluoromethyl)benzyloxy)biphenyl-4-yl]propanoic acid,
70. 3-[2-(3-Isopropoxybenzyloxy)-3′-({[3-(4-methoxyphenyl)propionyl](methyl)amino}methyl)biphenyl-4-yl]propanoic acid,
71. 3-[2′-(3-Methoxybenzyloxy)-5′-({[3-(3-methoxyphenyl)propionyl](methyl)amino}methyl)biphenyl-4-yl]propanoic acid,
72. 3-{2′-Cyclohexylmethoxy-5′-[((hexanoyl)(methyl)amino)methyl]biphenyl-4-yl}propanoic acid,
73. 3-{4′-Ethoxy-3′-[((hexanoyl)(methyl)amino)methyl]-2-propoxybiphenyl-4-yl}propanoic acid,
74. 3-{3′-[((Hexanoyl)(methyl)amino)methyl]-3,5-dimethoxybiphenyl-4-yl}propanoic acid,
75. 3-[3,5-Diethoxy-3′-({[3-(3-methoxyphenyl)propionyl](methyl)amino}methyl)biphenyl-4-yl]propanoic acid,
76. 3-[3′-({[3-(4-Methoxyphenyl)propionyl](methyl)amino}methyl)-3-propoxybiphenyl-4-yl]propanoic acid,
77. 3-{3-Cyclopropylmethoxy-3′-[((hexanoyl)(methyl)amino)methyl]biphenyl-4-yl}propanoic acid,
78. 3-{3-Ethoxy-4′-fluoro-3′-[((hexanoyl)(methyl)amino)methyl]biphenyl-4-yl}propanoic acid,
79. 3-[3′-[((Hexanoyl)(methyl)amino)methyl]-3-(4,4,4-trifluorobutoxy)biphenyl-4-yl]propanoic acid,
80. 3-{3-Benzyloxy-3′-[((hexanoyl)(methyl)amino)methyl]biphenyl-4-yl}propanoic acid,
81. 3-[3′-({[3-(4-Methoxyphenyl)propionyl](methyl)amino}methyl)-3-(3-(trifluoromethyl)benzyloxy)biphenyl-4-yl]propanoic acid,
82. 3-[3′-({[3-(3-Methoxyphenyl)propionyl](methyl)amino}methyl)-3,5-dipropylbiphenyl-4-yl]propanoic acid,
83. 3-{3-(2,2-Dimethylpropyl)-3′-[((hexanoyl)(methyl)amino)methyl]biphenyl-4-yl}propanoic acid,
84. 3-{3′-[((Hexanoyl)(methyl)amino)methyl]-3,5-dimethylbiphenyl-4-yl}propanoic acid,
85. 3-{3-[((Hexanoyl)(methyl)amino)methyl]-4″-methoxy-1,1′;3′,1″-terphenyl-4′-yl}propanoic acid,
86. 3-{3-[((Hexanoyl)(methyl)amino)methyl]-3″-methoxy-1,1′;2′,1″-terphenyl-4′-yl}propanoic acid,
87. 3-[3-({[3-(3-Methoxyphenyl)propionyl](methyl)amino}methyl)-3″-trifluoromethyl-1,1′;2′,1″-terphenyl-4′-yl]propanoic acid,
88. 3-{3′-[((Hexanoyl)(methyl)amino)methyl]-2-[2-(3-isopropoxyphenyl)ethyl]biphenyl-4-yl}propanoic acid,
89. 3-{3′-[((Hexanoyl)(methyl)amino)methyl]-2-[(pyridin-3-ylmethyl)amino]biphenyl-4-yl}propanoic acid,
90. 3-[3′-[((Hexanoyl)(methyl)amino)methyl]-3-(2-methoxyethylamino)biphenyl-4-yl]propanoic acid,
91. Methyl 3-{3,5-diethyl-3′-[((hexanoyl)(methyl)amino)methyl]biphenyl-4-yl}propanoate,
92. Methyl 3-[3′-{[methyl(3-phenoxybenzoyl)amino]-methyl}-2-(3-(trifluoromethyl)benzyloxy)biphenyl-4-yl]propanoate,
93. Methyl 3-[3′-{[(3-(biphenyl-4-yl)propionyl)(methyl)amino]methyl}-2-(3-methoxybenzyloxy)biphenyl-4-yl]propanoate,
94. Ethyl 3-[3′-({[3-(3-methoxyphenyl)propionyl](methyl)amino}methyl)-2-(4,4,4-trifluorobutoxy)biphenyl-4-yl]propanoate,
95. N-[4′-(2-(Hydroxycarbamoyl)ethyl)-4″-methoxy-1,1′;3′,1″-terphenyl-3-ylmethyl](methyl)hexanamide.

The compounds of formula (I) which are more particularly preferred are those which satisfy at least one of the following conditions:

R1 is a hydroxyl radical;

R2 and R7 are each an alkoxy or aryloxy radical, an alkylamino radical or a polyether radical;

R3 and R8 are each a hydrogen atom;

R4 is a lower alkyl radical having from 1 to 4 carbon atoms;

R5 is an alkyl radical having from 3 to 8 carbon atoms or an aryl radical;

Y is an oxygen atom;

the V—W bond is a C—C single or double bond.

In particular, more preferred are the compounds of general formula (I) satisfying all of the following conditions:

R1 is a hydroxyl radical;

R2 and R7 are each an alkoxy or aryloxy radical, an alkylamino radical or a polyether radical;

R3 and R8 are each a hydrogen atom;

R4 is a lower alkyl radical having from 1 to 4 carbon atoms;

R5 is an alkyl radical having from 3 to 8 carbon atoms or an aryl radical;

Y is an oxygen atom;

the V—W bond is a C—C single or double bond.

The present invention also features processes for the preparation of the compounds of formula (I), in particular according to the reaction schemes shown in the FIGURE of Drawing.

Route 1-2-3-4-5-6-7-8-9:

The amides 3 can be obtained by treating the corresponding amines 2 with acyl halides 1 possessing the desired chain. For example, octanoyl chloride can react with methylamine to provide the corresponding amide.

The amides 3 can react with benzyl halides 4 in the presence of a base, such as NaH, for example, to give the compounds 5.

The boronic ester 6 can be obtained by treating compound 5 with bis(pinacolato)diboron, for example in the presence of a palladium-based catalyst, such as [1,1′-bis(diphenylphosphino)ferrocene]dichloropalladium(II). Its corresponding boronic acid (R=H) can be obtained by employing conventional conditions, such as, for example, the use of tert-butyllithium, followed by addition to trimethyl borate.

The aryl halides or triflates 11 can be obtained commercially or else can be synthesized by a Wittig reaction by treating the suitably substituted aldehydes 10 with methyl (triphenylphosphoranylidene)acetate, for example.

A palladium coupling of Suzuki type from the boronate 6 and the appropriate reactant 11 (aryl bromide, iodide, chloride or triflate) makes it possible to obtain the compound exhibiting the aryl-aryl sequence 7.

The acid functional group of the compound 8 can be obtained from 7 by saponification, if R6 is an alkyl chain, with a base, such as sodium hydroxide, or by hydrogenolysis, if R6 is a benzyl.

The compound 9 can be obtained by hydrogenation, if 8 has V—W═CH═CH, under conventional hydrogenation conditions, such as, for example, hydrogen catalyzed by palladium-on-charcoal.

The compound 17 can be obtained from the acid 9 by treatment with oxalyl chloride, for example, followed by reaction with hydroxylamine.

The compound 17 can be obtained from the ester 18 by treatment with hydroxylamine.

The compound 7 can be subjected to a deprotection/protection sequence if R5=O-(t-Bu). In this case, treatment with trifluoroacetic acid, for example, followed by reaction with an acyl chloride, can result in the compounds where R5=alkyl.

In the case where R2=OBn, a hydrogenation reaction, in the presence of palladium-on-charcoal, for example, and under a hydrogen atmosphere, can be carried out on the compound 7 to provide the compounds 18 in which R2=OH.

The compounds 19 can be obtained by alkylation of the phenol under conventional conditions, such as, for example, in the presence of an alkyl halide and of potassium carbonate.

The acid functional group of the compound 9 can be obtained from 18 or 19 by saponification, if R6 is an alkyl chain, with a base, such as sodium hydroxide.

Route 10-11-12-16-14-7-8-9:

The compounds 11 can be obtained from the corresponding aldehydes 10 by a Wittig reaction with methyl (triphenylphosphoranylidene)acetate.

The boronic ester 12 can be obtained by treating the compound 11 with bis(pinacolato)diboron, for example in the presence of a palladium-based catalyst, such as [1,1′-bis(diphenylphosphino)ferrocene]dichloropalladium(II).

A palladium coupling of Suzuki type from the boronate 12 and the appropriate reactant 13 (bromide, iodide, chloride) makes it possible to obtain the compound exhibiting the aryl-aryl sequence 14.

The compound 14 can be subjected to a conventional acylation reaction with an acyl halide in order to provide the compound 7 in which R4=H.

A palladium coupling of Suzuki type from the boronate 12 and the appropriate reactant 5 (bromide, iodide, chloride) makes it possible to obtain the compounds exhibiting the aryl-aryl sequence 7.

Route 6-10-15-7-8-9:

A palladium coupling of Suzuki type from the boronate 6 (or the corresponding boronic acid) and the appropriate reactant 10 (aryl bromide, iodide, chloride or triflate) makes it possible to obtain the compound exhibiting the aryl-aryl sequence 15.

The compound 7 can be obtained by a Wittig reaction by treating the corresponding compound 15 with methyl (triphenylphosphoranylidene)acetate, for example.

Route 5-16-15-7-8-9:

A palladium coupling of Suzuki type from the boronic acid 16 and the compound 5 (aryl bromide, iodide, chloride or triflate) makes it possible to obtain the compound exhibiting the aryl-aryl sequence 15.

The compounds according to the invention exhibit modulatory properties with regard to receptors of PPAR type. This activity on PPARα, δ and γ receptors is measured in a transactivation test and quantified by the dissociation constant Kdapp (apparent), as described in Example 17.

The preferred compounds of the present invention exhibit a dissociation constant of less than or equal to 5,000 nM and advantageously of less than or equal to 1,000 nM.

Preferably, the compounds are modulators of specific receptors of PPARγ type, that is to say that they exhibit a ratio of the Kdapp for the PPARα or PPARδ receptors to the Kdapp for the PPARγ receptors of greater than or equal to 10. Preferably, this PPARα/PPARγ or PPARδ/PPARγ ratio is greater than or equal to 50 and more advantageously greater than or equal to 100.

The present invention also features administration of the compounds of formula (I) as medicaments.

The compounds according to the invention are particularly useful in the following fields of treatment, whether regime or regimen:

1) for treating dermatological conditions or afflictions linked to a disorder of keratinization involving differentiation and proliferation, in particular for treating acne vulgaris, comedonic or polymorphic acne, acne rosacea, nodulocystic acne, acne conglobata, senile acne or secondary acnes, such as solar, drug or occupational acne,

2) for treating other types of disorders of keratinization, in particular ichthyoses, ichthyosiform conditions, Darier's disease, palmoplantar keratoderma, leucoplakia and leucoplakiform conditions, or cutaneous or mucosal (oral) lichen,

3) for treating other dermatological conditions, disorders or afflictions having an inflammatory immunoallergic component, with or without cell proliferation disorder, and, in particular, all forms of psoriasis, whether cutaneous, mucosal or ungual, and even psoriatic rheumatism, or, alternatively, cutaneous atopy, such as eczema, or respiratory atopy or, alternatively, gingival hypertrophy,

4) for treating all dermal or epidermal proliferations, whether they are benign or malignant and whether they are or are not of viral origin, such as common warts, flat warts and epidermodysplasia verruciformis, florid or oral papillomatoses, T lymphoma, and the proliferations which can be induced by ultraviolet radiation, in particular in the case of basal cell and prickle cell epithelioma, and also all precancerous skin lesions, such as keratoacanthomas,

5) for treating other dermatological disorders, conditions or afflictions, such as immune dermatoses, such as lupus erythematosus, immune bullous diseases and collagen diseases, such as scleroderma,

6) in the treatment of dermatological or general conditions having an immunological component,

7) in the treatment of skin disorders due to exposure to UV radiation, and also for repairing or combating skin aging, whether photoinduced or chronologic, or for reducing actinic keratoses and pigmentations, or any pathology associated with chronologic or actinic aging, such as xerosis,

8) for combating disorders of the sebaceous function, such as hyperseborrhoea of acne or simple seborrhoea,

9) for preventing or treating disorders of cicatrization or for preventing or repairing stretch marks,

10) in the treatment of disorders of pigmentation, such as hyperpigmentation, melasma, hypopigmentation or vitiligo,

11) in the treatment of conditions of the metabolism of lipids, such as obesity, hyperlipidaemia or non-insulin-dependent diabetes,

12) in the treatment of inflammatory conditions, such as arthritis,

13) in the treatment or prevention of cancerous or precancerous conditions,

14) in the prevention or treatment of alopecia of various origins, in particular alopecia due to chemotherapy or to radiation,

15) in the treatment of disorders of the immune system, such as asthma, type I diabetes mellitus, multiple sclerosis or other selective dysfunctions of the immune system,

16) in the treatment of conditions of the cardiovascular system, such as arteriosclerosis or hypertension.

The present invention also features pharmaceutical/cosmetic compositions comprising, formulated into a physiologically acceptable medium, at least one compound of formula (I) as defined above.

This invention also features administration of the compounds of formula (I) for the treatment of the abovementioned conditions, disorders or afflictions, in particular for regulating and/or restoring the metabolism of skin lipids.

The compositions according to the invention can be administered orally, enterally, parenterally, topically or ocularly. Preferably, the pharmaceutical composition is packaged in a form suitable for topical application.

Orally, the composition, more particularly the pharmaceutical composition, can be provided in the form of tablets, including sugar-coated tablets, hard gelatin capsules, syrups, suspensions, solutions, powders, granules, emulsions or lipid or polymeric microspheres or nanospheres or vesicles which make possible controlled release. Parenterally, the composition can be provided in the form of solutions or suspensions for infusion or for injection.

The compounds according to the invention are generally administered at a daily dose of approximately 0.001 mg/kg to 100 mg/kg of body weight, taken 1 to 3 times.

The compounds are administered systemically at a concentration generally of from 0.001% to 10% by weight, preferably from 0.01% to 1% by weight, with respect to the weight of the composition.

Topically, the pharmaceutical compositions according to the invention are more particularly useful for the treatment of the skin and mucous membranes and can be provided in the form of salves, creams, milks, ointments, powders, impregnated pads, solutions, gels, sprays, lotions or suspensions. They can also be provided in the form of lipid or polymeric microspheres or nanospheres or vesicles or of polymeric patches and of hydrogels which make possible controlled release. The topical compositions can be provided in the anhydrous form, in the aqueous form or in the form of an emulsion.

The compounds are administered topically at a concentration generally of from 0.001% to 10% by weight, preferably from 0.01% to 1% by weight, with respect to the total weight of the composition.

The compounds of formula (I) according to the invention also have an application in the cosmetics field, in particular in body and hair hygiene and more particularly for regulating and/or restoring the metabolism of skin lipids. In comparison with the products known previously, these compounds of formula (I) have the advantage of additionally exhibiting other advantageous properties, in particular anti-inflammatory or soothing properties, which makes them less irritating and therefore better tolerated compounds.

This invention thus features cosmetic compositions comprising, formulated into a physiologically acceptable vehicle, at least one of the compounds of formula (I) for body or hair hygiene.

The cosmetic compositions according to the invention, comprising, in a cosmetically acceptable vehicle, at least one compound of formula (I) or one of its optical or geometrical isomers or one of its salts, can be provided in particular in the form of a cream, a milk, a lotion, a gel, lipid or polymeric microspheres or nanospheres or vesicles, a soap or a shampoo.

The concentration of compound of formula (I) in the cosmetic compositions is from 0.001% to 3% by weight, with respect to the total weight of the composition.

The compositions as described above can, of course, additionally comprise inert or even pharmacodynamically active additives or combinations of these additives and, in particular: wetting agents; depigmenting agents, such as hydroquinone, azelaic acid, caffeic acid or kojic acid; emollients; moisturizing agents, such as glycerol, polyethylene glycol (PEG) 400, thiamorpholinone and its derivatives, or urea; anti-seborrhoeic or anti-acne agents, such as S-carboxymethylcysteine, S-benzylcysteamine, their salts or their derivatives, or benzoyl peroxide; anti-fungal agents, such as ketoconazole or 4,5-polymethylene-3-isothiazolidones; anti-bacterials; carotenoids and in particular β-carotene; anti-psoriatic agents, such as anthralin and its derivatives; eicosa-5,8,11,14-tetraynoic and eicosa-5,8,11-triynoic acids, their esters and amides; and, finally, retinoids. The compounds of formula (I) can also be combined with vitamins D or their derivatives, with corticosteroids, with agents for combating free radicals, with α-hydroxy or α-keto acids or their derivatives, or with ion-channel blockers.

These compositions can also comprise flavor enhancers, preservatives, such as esters of para-hydroxybenzoic acid, stabilizing agents, moisture-regulating agents, pH-regulating agents, agents for modifying osmotic pressure, emulsifying agents, UV-A and UV-B screening agents, or antioxidants, such as α-tocopherol, butylated hydroxyanisole or butylated hydroxytoluene.

Of course, one skilled in this art will take care to select the optional compound or compounds to be added to these compositions so that the advantageous properties intrinsically associated with the present invention are not, or not substantially, detrimentally affected by the envisaged addition.

In order to further illustrate the present invention and the advantages thereof, the following examples of specific active compounds are given, as are the biological activities of such compounds and specific formulations thereof, it being understood that same are intended only as illustrative and in nowise limitative. In said examples to follow, all parts and percentages are given by weight, unless otherwise indicated.

### EXAMPLE 1

### Synthesis of 3-(3′-[((methyl)(octanoyl)amino)methyl]biphenyl-4-yl)acrylic acid

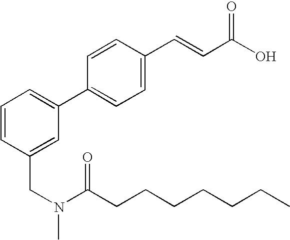

### a. Preparation of N-methyloctanamide

115 ml of triethylamine are added to a solution of 25 g (0.37 mol, 1 eq.) of methylamine hydrochloride in 125 ml of dichloromethane. 70 ml (0.41 mol, 1.1 eq.) of octanoyl chloride are slowly added at 0° C. The reaction mixture is stirred at ambient temperature for 3 hours and then filtered, and 100 ml of water are added to the filtrate. The organic phase is separated by settling, dried over sodium sulfate and evaporated. 61 g of N-methyloctanamide are obtained with a quantitative yield.

### b. Preparation of N-methyl-N-(3-bromobenzyl)octanamide

5 g (31.8 mmol, 1 eq) of N-methyloctanamide are added at 0° C. to a suspension of 1.4 g (35 mmol, 1.1 eq.) of sodium hydride (60% in oil) in 60 ml of tetrahydrofuran. The reaction mixture is stirred at ambient temperature for 30 minutes and then a solution of 8.9 g (35 mmol, 1.1 eq.) of 3-bromobenzyl bromide in 15 ml of tetrahydrofuran is added. The mixture is stirred at ambient temperature for 16 hours. The reaction is halted by the addition of 100 ml of water and then extraction is carried out with ethyl acetate. The organic phases are combined and dried over sodium sulfate. The solvents are evaporated and then the residue is chromatographed on silica gel (heptane/ethyl acetate 75/25). 7.4 g of N-methyl-N-(3-bromobenzyl)octanamide are obtained. Yield=71%

### c. Preparation of methyl[3-(4,4,5,5-tetramethyl-1,3,2-dioxaborolan-2-yl)benzyl]octanamide

1.0 g (3.98 mmol, 1.3 eq.) of bis(pinacolato)diboron is added to a mixture of 1.0 g (3.06 mmol, 1 eq.) of N-methyl-N-(3-bromobenzyl)octanamide and of 900 mg (9.18 mmol, 3 eq.) of potassium acetate in 10 ml of dimethylformamide in the presence of 111 mg (0.15 mmol, 5 mol %) of [1,1′-bis(diphenylphosphino)ferrocene]dichloropalladium(II) (PdCl₂dppf). The mixture is stirred at 80° C. for 2 hours. The reaction is halted by the addition of 20 ml of water and then extraction is carried out with ethyl acetate. The organic phases are combined and dried over sodium sulfate. The solvents are evaporated and then the residue is chromatographed on silica gel (heptane/ethyl acetate 80/20). 1.0 g of methyl[3-(4,4,5,5-tetramethyl-1,3,2-dioxaborolan-2-yl)benzyl]octanamide is obtained in the form of an oil. Yield=88%

### d. Preparation of ethyl 3-(3′-[((methyl)(octanoyl)amino)methyl]biphenyl-4-yl}acrylate

30 mg (0.02 mmol, 5 mol %) of palladiumtetrakis(triphenylphosphine) are added to a solution of 200 mg (0.53 mmol, 1.0 eq.) of methyl[3-(4,4,5,5-tetramethyl-1,3,2-dioxaborolan-2-yl)benzyl]octanamide and 204 mg (0.80 mmol, 1.5 eq.) of ethyl 4-bromocinnamate in 3 ml of a mixture of dimethylformamide and of a 2M potassium phosphate solution (6/1). The reaction mixture is stirred at 80° C. for 2 hours. The reaction is halted by the addition of 5 ml of water and then extraction is carried out with ethyl acetate. The organic phases are combined and dried over sodium sulfate. The solvents are evaporated and then the residue is chromatographed on silica gel (heptane/ethyl acetate 80/20). 145 mg of ethyl 3-(3′-[((methyl)(octanoyl)amino)methyl]biphenyl-4-yl}acrylate are obtained in the form of an oil. Yield=63%

### e. Synthesis of 3-{3′-[((methyl)(octanoyl)amino)methyl]biphenyl-4-yl)acrylic acid

300 mg (7.5 mmol, 22 eq.) of sodium hydroxide are added to a solution of 145 mg (0.34 mmol, 1 eq.) of ethyl 3-(3′-[((methyl)(octanoyl)amino)methyl]biphenyl-4-yl}acrylate in 3 ml of tetrahydrofuran/methanol (9/1). The reaction mixture is stirred at ambient temperature for 2 hours. The reaction is halted by the addition of 20 ml of water and 3 ml of acetic acid and then extraction is carried out with ethyl acetate. The organic phases are combined and dried over sodium sulfate. The solvents are evaporated and then the residue is crystallized from pentane/dichloromethane. 110 mg of 3-{3′-[((methyl)(octanoyl)amino)methyl]biphenyl-4-yl)acrylic acid are obtained in the form of a white powder. (M.p.=124-125° C.) Yield=78%

¹H NMR (CDCl₃, 400 MHz): 0.88 (m, 3H), 1.30 (m, 8H), 1.71 (m, 2H), 2.44 (t, J=7.6 Hz, 2H), 2.99 & 3.03 (2s (rotamers), 3H), 4.65 & 4.70 (2s (rotamers), 2H), 6.51 (2d (rotamers), J=15.9 Hz, 1H), 7.22 (2d (rotamers), J=6.6 Hz, 1H), 7.41-7.66 (m, 7H), 7.82 (2d (rotamers), J=15.9 Hz, 1H).

### EXAMPLE 2

### Synthesis of 3-{3′-[((methyl)(octanoyl)amino)methyl]biphenyl-4-yl}propanoic acid

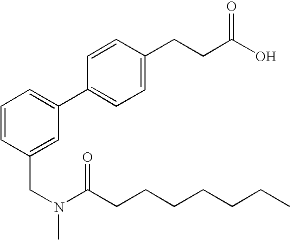

### a. Preparation of ethyl 3-{3′-[((methyl)(octanoyl)amino)methyl]biphenyl-4-yl}propanoate

A solution of 169 mg (0.40 mmol, 1 eq.) of ethyl 3-(3′-[((methyl)(octanoyl)amino)methyl]biphenyl-4-yl}acrylate (prepared as is 1d) in 2 ml of methanol in the presence of 50 mg of 10% palladium-on-charcoal is stirred at ambient temperature for 2 hours under a hydrogen atmosphere. The palladium is filtered off and then the solvents are evaporated. The residue is chromatographed on silica gel (heptane/ethyl acetate 80/20). 136 mg of ethyl 3-{3′-[((methyl)(octanoyl)amino)methyl]biphenyl-4-yl}propanoate are obtained in the form of an oil. Yield=80%

### b. Synthesis of 3-{3′-[((methyl)(octanoyl)amino)methyl]biphenyl-4-yl}propanoic acid

300 mg (7.5 mmol, 23 eq.) of sodium hydroxide are added to a solution of 136 mg (0.32 mmol, 1 eq.) of ethyl 3-{3′-[((methyl)(octanoyl)amino)methyl]biphenyl-4-yl}propanoate in 3 ml of tetrahydrofuran/methanol (9/1). The reaction mixture is stirred at ambient temperature for 2 hours. The reaction is halted by the addition of 20 ml of water and 3 ml of acetic acid and then the extraction is carried out with ethyl acetate. The organic phases are combined and dried over sodium sulfate. The solvents are evaporated and then the residue is crystallized from pentane/dichloromethane. 85 mg of 3-{3′-[((methyl)(octanoyl)amino)methyl]biphenyl-4-yl}propanoic acid are obtained in the form of a white powder. (M.p.=91° C.) Yield=65%

¹H NMR (CDCl₃, 400 MHz): 0.88 (m, 3H), 1.30 (m, 8H), 1.70 (m, 2H), 2.41 (t, J=7.5 Hz, 2H), 2.74 (m, 2H), 2.97 & 3.00 (2s (rotamers), 3H), 3.01 (m, 2H), 4.61 &4.68 (2s (rotamers), 2H), 6.51 (2d e, J=15.9 Hz, 1H), 7.14 & 7.22 (2d (rotamers), J=7.4 Hz, 1H), 7.28-7.54 (m, 7H).

### EXAMPLE 3

### Synthesis of 3-{3-fluoro-3′-[((methyl)(octanoyl)amino)methyl]biphenyl-4-yl}acrylic acid

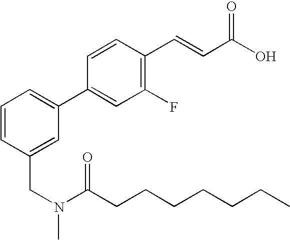

### a. Preparation of methyl 3-(4-bromo-2-fluorophenyl)acrylate

2.17 g (6.5 mmol, 1.2 eq.) of methyl (triphenylphosphoranylidene)acetate are added to a solution of 1.1 g (5.4 mmol, 1.0 eq.) of 4-bromo-2-fluorobenzaldehyde in 10 ml of toluene. The reaction mixture is stirred at 80° C. for 1 hour. The reaction is halted by the addition of 20 ml of water and then extraction is carried out with ethyl acetate. The organic phases are combined and dried over sodium sulfate. The solvents are evaporated and then the residue is chromatographed on silica gel (heptane/ethyl acetate 80/20). 1.1 g of methyl 3-(4-bromo-2-fluorophenyl)acrylate are obtained in the form of a white solid. Yield=78%

### b. Preparation of methyl 3-{3-fluoro-3′-[((methyl)(octanoyl)amino)methyl]biphenyl-4-yl}acrylate

500 mg (1.93 mmol, 1.0 eq.) of methyl 3-(4-bromo-2-fluorophenyl)acrylate and 721 mg (1.93 mmol, 1.0 eq.) of methyl[3-(4,4,5,5-tetramethyl-1,3,2-dioxaborolan-2-yl)benzyl]octanamide (prepared as in 1c) are dissolved in 12 ml of a mixture of dimethylformamide and of a 2M potassium phosphate solution (5/1). 111 mg (0.09 mmol, 5 mol %) of palladiumtetrakis(triphenylphosphine) are added and then the reaction mixture is stirred at 80° C. for 2 hours. The reaction is halted by the addition of 30 ml of water and then extraction is carried out with ethyl acetate. The organic phases are combined and dried over sodium sulfate. The solvents are evaporated and then the residue is chromatographed on silica gel (heptane/ethyl acetate 80/20). 615 mg of methyl 3-{3-fluoro-3′-[((methyl)(octanoyl)amino)methyl]biphenyl-4-yl}acrylate are obtained. Yield=75%

### c. Synthesis of 3-{3-fluoro-3′-[((methyl)(octanoyl)amino)methyl]biphenyl-4-yl}acrylic acid

500 mg (12.5 mmol, 9 eq.) of sodium hydroxide are added to a solution of 615 mg (1.44 mmol, 1 eq.) of methyl 3-{3-fluoro-3′-[((methyl)(octanoyl)amino)methyl]biphenyl-4-yl}acrylate in 5 ml of tetrahydrofuran/methanol (9/1). The reaction mixture is stirred at ambient temperature for 2 hours. The reaction is halted by the addition of 20 ml of water and 3 ml of acetic acid and then extraction is carried out with ethyl acetate. The organic phases are combined and dried over sodium sulfate. The solvents are evaporated and then the residue is chromatographed on silica gel (dichloromethane/methanol 90/10). The oil obtained is crystallized from pentane. 510 mg of 3-{3-fluoro-3′-[((methyl)(octanoyl)amino)methyl]biphenyl-4-yl}acrylic acid are obtained in the form of a white powder. (M.p.=91° C.) Yield=86%

¹H NMR (CDCl₃, 400 MHz): 0.88 (m, 3H), 1.30 (m, 8H), 1.72 (m, 2H), 2.45 (m, 2H), 3.00 & 3.03 (2s (rotamers), 3H), 4.65 & 4.69 (2s (rotamers), 2H), 6.59 & 6.61 (2d rotamers, J=16.1 Hz, 1H), 7.28-7.63 (m, 7H), 7.91 & 7.93 (2d rotamers, J=16.1 Hz, 1H).

### EXAMPLE 4

### Synthesis of 3-[3-fluoro-3′-[((methyl)(octanoyl)amino)methyl]biphenyl-4-yl)propanoic acid

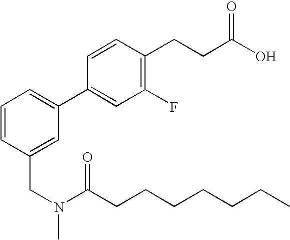

A solution of 200 mg (0.48 mmol, 1 eq.) of 3-{3-fluoro-3′-[((methyl)(octanoyl)amino)methyl]biphenyl-4-yl}acrylic acid (prepared as in 3c) in 3 ml of methanol in the presence of 50 mg of 10% palladium-on-charcoal is stirred at ambient temperature for 2 hours under a hydrogen atmosphere. The palladium is filtered off and then the solvents are evaporated. The residue is chromatographed on silica gel (dichloromethane/methanol 90/10). 154 mg of 3-{3-fluoro-3′-[((methyl)(octanoyl)amino)methyl]biphenyl-4-yl)propanoic acid are obtained. Yield=78%

¹H NMR (CDCl₃, 400 MHz): 0.88 (m, 3H), 1.30 (m, 8H), 1.70 (m, 2H), 2.41 (t, J=7.6 Hz, 2H), 2.74 (m, 2H), 2.97 & 3.00 (2s (rotamers), 3H), 3.05 (m, 2H), 4.62 & 4.67 (2s e, 2H), 7.18 & 7.49 (m, 7H).

### EXAMPLE 5

### Synthesis of 3-[3′-((octanoylamino)methyl)biphenyl-4-yl]acrylic acid

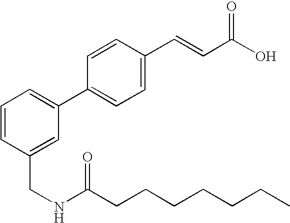

### a. Preparation of ethyl 3-[4-(4,4,5,5-tetramethyl-1,3,2-dioxaborolan-2-yl)phenyl]acrylate

1.1 g (4.3 mmol, 1.1 eq.) of bis(pinacolato)diboron are added to a mixture of 1.0 g (3.9 mmol, 1 eq.) of ethyl 4-bromocinnamate and 1.1 g (11.7 mmol, 3 eq.) of potassium acetate in the presence of 142 mg (0.19 mmol, 5 mol %) of [1,1′-bis(diphenylphosphino)ferrocene]dichloropalladium(II) (PdCl₂dppf) in 10 ml of dimethylformamide. The mixture is stirred at 70° C. for 2 hours. The reaction is halted by the addition of 20 ml of water and then extraction is carried out with ethyl acetate. The organic phases are combined and dried over sodium sulfate. The solvents are evaporated and then the residue is chromatographed on silica gel (heptane/ethyl acetate 90/10). 1.15 g of ethyl 3-[4-(4,4,5,5-tetramethyl-1,3,2-dioxaborolan-2-yl)phenyl]acrylate are obtained in the form of an oil. Yield=98%

### b. Preparation of ethyl 3-[3′-((octanoylamino)methyl)biphenyl-4-yl]acrylate

124 mg (0.10 mmol, 5 mol %) of palladiumtetrakis(triphenylphosphine) are added to a solution of 500 mg (2.14 mmol, 1.0 eq.) of 3-iodobenzylamine and 712 mg (2.36 mmol, 1.1 eq) of ethyl 3-[4-(4,4,5,5-tetramethyl-1,3,2-dioxaborolan-2-yl)phenyl]acrylate in 6 ml of a mixture of dimethylformamide and of a 2M potassium phosphate solution (5/1). The reaction mixture is stirred at 80° C. for 1 hour. The reaction is halted by the addition of 15 ml of water and then extraction is carried out with ethyl acetate. The organic phases are combined and dried over sodium sulfate. The solvents are evaporated and then the residue is dissolved in 25 ml of tetrahydrofuran. 2 ml of triethylamine are added, along with 0.5 ml (2.9 mmol, 1.4 eq.) of octanoyl chloride. The reaction mixture is stirred at ambient temperature for 1 hour. The reaction is halted by the addition of 20 ml of water and then extraction is carried out with ethyl acetate. The organic phases are combined and dried over sodium sulfate. The solvents are evaporated and then the residue is chromatographed on silica gel (heptane/ethyl acetate 70/30 down to 50/50). 430 mg of ethyl 3-[3′-((octanoylamino)methyl)biphenyl-4-yl]acrylate are obtained. Yield=49%

### c. Synthesis of 3-[3′-((octanoylamino)methyl)biphenyl-4-yl]acrylic acid

400 mg (10 mmol, 9 eq.) of sodium hydroxide are added to a solution of 430 mg (1.04 mmol, 1 eq.) of ethyl 3-[3′-((octanoylamino)methyl)biphenyl-4-yl]acrylate in 8 ml of tetrahydrofuran/methanol (9/1). The reaction mixture is stirred at ambient temperature for 2 hours. The reaction is halted by the addition of 20 ml of water and 3 ml of acetic acid and then extraction is carried out with ethyl acetate. The organic phases are combined and dried over sodium sulfate. The solvents are evaporated and then the residue is chromatographed on silica gel (dichloromethane/methanol 80/20). The oil obtained is crystallized from heptane/dichloromethane. 230 mg of 3-[3′-((octanoylamino)methyl)biphenyl-4-yl]acrylic acid are obtained in the form of a white powder. (M.p.=176° C.) Yield=58%

¹H NMR (d₆-DMSO, 400 MHz): 0.83 (m, 3H), 1.24 (m, 8H), 1.53 (m, 2H), 2.15 (t, J=7.6 Hz, 2H), 4.33 (d, J=5.4 Hz, 2H), 6.58 (d, J=15.9 Hz, 1H), 7.26 (d, J=7.0 Hz, 1H), 7.42 (t, J=7.8 Hz, 1H), 7.58 (m, 3H), 7.69 (d, J=8.0 Hz, 2H), 7.78 (d, J=7.7 Hz, 2H), 8.35 (s, 1H).

### EXAMPLE 6

### Synthesis of 3-[3′-((octanoylamino)methyl)biphenyl-4-yl]propanoic acid

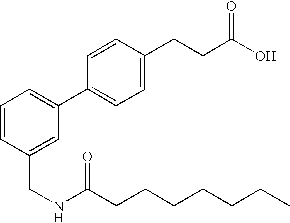

A solution of 100 mg (0.26 mmol, 1 eq.) of 3-[3′-((octanoylamino)methyl)biphenyl-4-yl]acrylic acid (prepared as in 5c) in 4 ml of methanol in the presence of 50 mg of 10% palladium-on-charcoal is stirred at ambient temperature for 6 hours under a hydrogen atmosphere. The palladium is filtered off and the solvents are evaporated. The residue is crystallized from dichloromethane/heptane and 65 mg of 3-[3′-((octanoylamino)methyl)biphenyl-4-yl]propanoic acid are obtained in the form of a white powder. (M.p.=124-125° C.) Yield=65%

¹H NMR (d₆-DMSO, 400 MHz): 0.84 (m, 3H), 1.24 (m, 8H), 1.53 (m, 2H), 2.14 (t, J=6.7 Hz, 2H), 2.57 (t, J=7.0 Hz, 2H), 2.86 (t, J=6.7 Hz, 2H), 4.33 (d, J=5 Hz, 2H), 7.21 (d, J=6.9 Hz, 1H), 7.32 (d, J=7.4 Hz, 2H), 7.39 (m, 1H), 7.53 (m, 4H), 8.33 (s, 1H), 12.11 (s, 1H).

### EXAMPLE 7

### Synthesis of 3-{3-hydroxy-3′-[((methyl)(octanoyl)amino)methyl]biphenyl-4-yl}propanoic acid

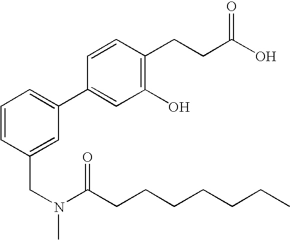

### a. Preparation of methyl 3-(2-benzyloxy-4-iodophenyl)acrylate

1.18 g (3.55 mmol, 1.2 eq.) of methyl (triphenylphosphoranylidene)acetate are added to a solution of 1.0 g (2.95 mmol, 1.0 eq.) of 2-benzyloxy-4-iodobenzaldehyde in 20 ml of toluene. The reaction mixture is stirred at 80° C. for 2 hours. The reaction is halted by the addition of 20 ml of water and then extraction is carried out with ethyl acetate. The organic phases are combined and dried over sodium sulfate. The solvents are evaporated and then the residue is filtered through silica gel (heptane/ethyl acetate 50/50). The oil obtained is crystallized from pentane and 830 mg of methyl 3-(2-benzyloxy-4-iodophenyl)acrylate are obtained in the form of a white solid. Yield=71%

### b. Preparation of methyl 3-{3-benzyloxy-3′-[((methyl)(octanoyl)amino)methyl]biphenyl-4-yl}acrylate

500 mg (1.26 mmol, 1.0 eq.) of methyl 3-(2-benzyloxy-4-iodophenyl)acrylate and 474 mg (1.26 mmol, 1.0 eq.) of methyl[3-(4,4,5,5-tetramethyl-1,3,2-dioxaborolan-2-yl)benzyl]octanamide (prepared as in 1c) are dissolved in 6 ml of a mixture of dimethylformamide and of a 2M potassium phosphate solution (5/1). 72 mg (0.06 mmol, 5 mol %) of palladiumtetrakis(triphenylphosphine) are added and then the reaction mixture is stirred at 80° C. for 2 hours. The reaction is halted by the addition of 30 ml of water and then extraction is carried out with ethyl acetate. The organic phases are combined and dried over sodium sulfate. The solvents are evaporated and then the residue is chromatographed on silica gel (heptane/ethyl acetate 80/20 down to 50/50). 530 mg of methyl 3-{3-benzyloxy-3′-[((methyl)(octanoyl)amino)methyl]biphenyl-4-yl]acrylate are obtained. Yield=82%

### c. Preparation of 3-{3-benzyloxy-3′-[((methyl)(octanoyl)amino)methyl]biphenyl-4-yl}acrylic acid

400 mg (10 mmol, 10 eq.) of sodium hydroxide are added to a solution of 530 mg (1.03 mmol, 1 eq.) of methyl 3-{3-benzyloxy-3′-[((methyl)(octanoyl)amino)methyl]biphenyl-4-yl}acrylate in 2 ml of tetrahydrofuran/methanol (9/1). The reaction mixture is stirred at ambient temperature for 2 hours. The reaction is halted by the addition of 20 ml of water and 3 ml of acetic acid and then extraction is carried out with ethyl acetate. The organic phases are combined and dried over sodium sulfate. The solvents are evaporated and then the residue is chromatographed on silica gel (dichloromethane/methanol 90/10). 405 mg of 3-{3-benzyloxy-3′-[((methyl)(octanoyl)amino)methyl]biphenyl-4-yl}acrylic acid are obtained. Yield=79%

### d. Synthesis of 3-{3-hydroxy-3′-[((methyl)(octanoyl)amino)methyl]biphenyl-4-yl}propanoic acid

A solution of 405 mg (0.81 mmol, 1 eq.) of 3-{3-benzyloxy-3′-[((methyl)(octanoyl)amino)methyl]biphenyl-4-yl}acrylic acid in 3 ml of methanol in the presence of 50 mg of 10% palladium-on-charcoal and of 20 μl of acetic acid is stirred at ambient temperature for 3 hours under a hydrogen atmosphere. The palladium is filtered off and then the solvents are evaporated. The residue is chromatographed on silica gel (dichloromethane/methanol 95/5). 135 mg of 3-{3-hydroxy-3′-[((methyl)(octanoyl)amino)methyl]biphenyl-4-yl}propanoic acid are obtained. Yield=40%

¹H NMR (CDCl₃, 400 MHz): 0.86 (m, 3H), 1.28 (m, 8H), 1.69 (m, 2H), 2.41 (t, J=7.7 Hz, 2H), 2.81 (m, 2H), 2.95 & 2.99 (2s (rotamers), 3H), 2.99 (m, 2H), 4.59 & 4.64 (2s e, 2H), 7.02-7.46 (m, 7H).

### EXAMPLE 8

### Synthesis of 3-{2-butoxy-3′-[((methyl)(octanoyl)amino)methyl]biphenyl-4-yl}propanoic acid

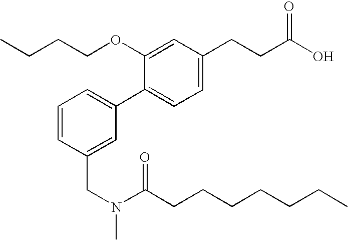

### a. Preparation of 3-hydroxy-4-iodobenzoic acid

21.0 g (0.52 mol, 1.05 eq.) of sodium hydroxide and then 78.7 g (0.52 mol, 1.05 eq.) of sodium iodide are added to a solution of 69.1 g (0.5 mol, 1 eq.) of 3-hydroxybenzoic acid in 700 ml of methanol. The reaction mixture is cooled to 0° C. and then aqueous sodium hypochlorite solution (0.52 mol, 1.05 eq.) is added dropwise. The reaction medium is stirred at 0-5° C. for 2 hours and then at ambient temperature overnight. The methanol is evaporated and then the reaction medium is acidified with a concentrated hydrochloric acid solution. The precipitated product is filtered off, washed with water and dried. 121 g of 3-hydroxy-4-iodobenzoic acid are obtained in the form of an off-white solid. Yield=92%

### b. Preparation of methyl 3-hydroxy-4-iodobenzoate

59 ml (1.10 mol, 2.4 eq.) of sulfuric acid are added to a solution of 121 g (0.458 mol, 1 eq.) of 3-hydroxy-4-iodobenzoic acid in 700 ml of methanol. The reaction mixture is heated at reflux for 6 days. The methanol is evaporated and then the reaction medium is poured into water and extracted with ethyl acetate. The organic phases are combined, washed with water and dried over sodium sulfate. The solvent is concentrated and the solid obtained is filtered off and dried. 88.56 g of methyl 3-hydroxy-4-iodobenzoate are obtained in the form of white crystals. Yield=70%

### c. Preparation of methyl 3-butoxy-4-iodobenzoate

In a manner analogous to Example 26b, by reaction of 21.5 ml (0.189 mol, 1.5 eq.) of 1-iodobutane and of 35.03 g (0.126 mol, 1 eq.) of methyl 3-hydroxy-4-iodobenzoate in 350 ml of methyl ethyl ketone in the presence of 52.24 g (0.378 mol, 3 eq.) of potassium carbonate at 85° C. for 2½ hours, 41.78 g of methyl 3-butoxy-4-iodobenzoate are obtained in the form of white crystals after washing with heptane. Yield=99%

### d. Preparation of (3-butoxy-4-iodophenyl)methanol

8.17 g (0.375 mol, 3 eq.) of lithium borohydride are added to a solution of 41.78 g (0.125 mol, 1 eq.) of methyl 3-butoxy-4-iodobenzoate in 210 ml of tetrahydrofuran. The reaction medium is heated at 60° C. for 2 hours and is then gently hydrolyzed in an ice-cold and saturated ammonium chloride solution. The reaction medium is neutralized with concentrated hydrochloric acid and then extracted with ethyl acetate. The organic phases are washed with water and dried over magnesium sulfate. The solvent is evaporated and 38.31 g of (3-butoxy-4-iodophenyl)methanol are obtained in the form of a whitish oil. Yield=100%

### e. Preparation of 3-butoxy-4-iodobenzaldehyde

89.5 g (0.875 mol, 7 eq.) of manganese dioxide are added to a solution of 38.30 g (0.125 mol, 1 eq.) of (3-butoxy-4-iodophenyl)methanol in 250 ml of dichloromethane. The reaction medium is stirred at ambient temperature for 18 hours and then filtered through silica gel. The solvent is evaporated and 29.61 g of 3-butoxy-4-iodobenzaldehyde are obtained in the form of an orange oil. Yield=78%

### f. Preparation of methyl (E)-3-(3-butoxy-4-iodophenyl)acrylate

65.08 g (0.195 mol, 2 eq.) of methyl (triphenylphosphoranylidene)acetate are added to a solution of 29.60 g (0.097 mol, 1 eq.) of 3-butoxy-4-iodobenzaldehyde in 360 ml of toluene. The reaction mixture is heated at reflux for 2 hours. The solvent is evaporated and the oil obtained is chromatographed on silica gel (heptane/dichloromethane 50/50). 30.47 g of methyl (E)-3-(3-butoxy-4-iodophenyl)acrylate are obtained in the form of pale yellow crystals. Yield=87%

### g. Preparation of methyl (E)-3-(2-butoxy-3′-[((methyl)(octanoyl)amino)methyl]biphenyl-4-yl}acrylate

4.5 mg (1 mol %) of palladium acetate and 14 mg (2 mol %) of biphenyldicyclohexylphosphine are added to a solution of 720 mg (2.0 mmol, 1 eq.) of methyl (E)-3-(3-butoxy-4-iodophenyl)acrylate (prepared in stage f) and of 971 mg (2.6 mmol, 1.3 eq.) of methyl[3-(4,4,5,5-tetramethyl-1,3,2-dioxoborolan-2-yl)benzyl]octanamide (prepared in Example 1c) in 12 ml of a mixture of dimethylformamide and of a 2M potassium phosphate solution (5/1). The mixture is heated at 90° C. for 2 hours. The reaction medium is hydrolyzed in an ammonium chloride solution and then extracted with ethyl acetate. The organic phases are combined, washed with water and dried over magnesium sulfate. The solvent is evaporated and then the residual oil is chromatographed on silica gel (heptane/ethyl acetate 75/25). 946 mg of methyl (E)-3-{2-butoxy-3′-[((methyl)(octanoyl)amino)methyl]biphenyl-4-yl}acrylate are obtained in the form of an orange-pink oil. Yield=98%

### h. Preparation of methyl 3-{2-butoxy-3′-[((methyl)(octanoyl)amino)methyl]biphenyl-4-yl}propanoate

A solution of 916 mg (1.91 mmol, 1 eq.) of methyl (E)-3-{2-butoxy-3′-[((methyl)(octanoyl)amino)methyl]biphenyl-4-yl}acrylate in 10 ml of methanol in the presence of 92 mg (10% by weight) of 10% palladium-on-charcoal is stirred at ambient temperature for 3½ hours under a hydrogen atmosphere. The palladium is filtered off through celite and then the solvent is evaporated. 902 mg of methyl 3-{2-butoxy-3′-[((methyl)(octanoyl)amino)methyl]biphenyl-4-yl}propanoate are obtained in the form of a yellowish oil. Yield=98%

### i. Synthesis of 3-{2-butoxy-3′-[((methyl)(octanoyl)amino)methyl]biphenyl-4-yl}propanoic acid

740 mg (18.5 mmol, 10 eq.) of sodium hydroxide are added to a solution of 891 mg (1.85 mmol, 1 eq.) of methyl 3-{2-butoxy-3′-[((methyl)(octanoyl)amino)methyl]biphenyl-4-yl}propanoate in 20 ml of methanol. The reaction mixture is stirred at ambient temperature for 24 hours. The reaction medium is evaporated to dryness, the residue is taken up in water, acidification is carried out with a 2N hydrochloric acid solution and extraction is carried out with ethyl acetate. The organic phases are combined, washed with water and dried over magnesium sulfate. The solvent is evaporated and the oil obtained is chromatographed on silica gel (FlashSmart column 35 g) eluted with dichloromethane/methanol (98/2). 725 mg of 3-{2-butoxy-3′-[((methyl)(octanoyl)amino)methyl]biphenyl-4-yl}propanoic acid are obtained in the form of a whitish oil. Yield=84%

¹H NMR (CDCl₃, 400 MHz): 0.86 (t, 3H), 0.91 (t, 3H), 1.24-1.40 (m, 10H), 1.67-1.70 (m, 4H), 2.39 (m, 2H), 2.72 (t, 2H), 2.93 (d, 3H), 2.96-3.0 (m, 2H), 3.92-3.96 (m, 2H), 4.56-4.63 (d, 2H), 6.82-6.87 (m, 2H), 7.10-7.20 (d, 1H), 7.23 (t, 1H), 7.33-7.46 (m, 3H).

### EXAMPLE 9

### Synthesis of 3-{2-butoxy-3′-[((hexanoyl)(methyl)amino)methyl]biphenyl-4-yl}propanoic acid

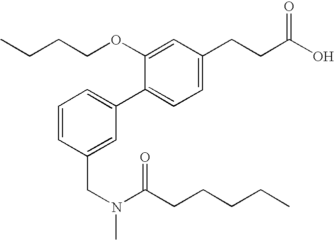

### a. Preparation of tert-butyl (3-bromobenzyl)carbamate

A solution of 25.0 g (0.11 mol, 1 eq.) of 3-bromobenzylamine hydrochloride and of 24.52 g (0.11 mol, 1 eq.) of di(tert-butyl)dicarbonate in 250 ml of dichloromethane in the presence of 15.6 ml (0.11 mol, 1 eq.) of triethylamine is stirred at ambient temperature for 16 hours. The reaction medium is washed with water and separated by settling, and the organic phase is dried over sodium sulfate. The solvent is evaporated and 32.41 g of tert-butyl (3-bromobenzyl)carbamate are obtained in the form of crystals. Yield=100%

### b. Preparation of tert-butyl (3-bromobenzyl)(methyl)carbamate

5.4 g (0.134 mol, 1.2 eq.) of 60% sodium hydride are added to a solution of 32.0 g (0.111 mol, 1 eq.) of tert-butyl (3-bromobenzyl)carbamate in 450 ml of dimethylformamide. The reaction medium is stirred at ambient temperature for 30 minutes and then 20.9 ml (0.335 mol, 3 eq.) of iodomethane are added. The reaction medium is stirred at ambient temperature for 20 hours, then hydrolyzed in water and extracted with ethyl acetate. The organic phases are combined, washed with a saturated sodium chloride solution and dried over sodium sulfate. The solvent is evaporated and 36.23 g of tert-butyl (3-bromobenzyl)(methyl)carbamate are obtained in the form of an orange oil. Yield=100%

### c. Preparation of tert-butyl methyl[3-(4,4,5,5-tetramethyl-1,3,2-dioxaborolan-2-yl)benzyl]carbamate

In a manner analogous to Example 1c, by reaction of 33.0 g (0.11 mol, 1 eq.) of tert-butyl (3-bromobenzyl)(methyl)carbamate, of 29.3 g (0.115 mol, 1.05 eq.) of bis(pinacolato)diboron and of 32.3 g (0.33 mol, 3 eq.) of potassium acetate in 500 ml of dimethylformamide in the presence of 3.6 g (4 mol %) of [1,1′-bis(diphenylphosphino)ferrocene]dichloropalladium(II) (PdCl₂dppf), 31.56 g of tert-butyl methyl[3-(4,4,5,5-tetramethyl-1,3,2-dioxaborolan-2-yl)benzyl]carbamate are obtained, after chromatography on silica gel (heptane/ethyl acetate 90/10), in the form of a green oil. Yield=83%

### d. Preparation of methyl (E)-3-{2-butoxy-3′-[((tert-butoxycarbonyl)(methyl)amino)methyl]biphenyl-4-yl}acrylate

In a manner analogous to Example 8g, by reaction of 3.6 g (10.0 mmol, 1 eq.) of methyl (E)-3-(3-butoxy-4-iodophenyl)acrylate (prepared in Example 80 and of 4.51 g (13 mmol, 1.3 eq.) of tert-butyl methyl[3-(4,4,5,5-tetramethyl-1,3,2-dioxaborolan-2-yl)benzyl]carbamate in 60 ml of a mixture of dimethylformamide and of a 2M potassium phosphate solution (5/1) in the presence of 22 mg (1 mol %) of palladium acetate and of 14 mg (2 mol %) of biphenyldicyclohexylphosphine, 3.87 g of methyl (E)-3-{2-butoxy-3′-[((tert-butoxycarbonyl)(methyl)amino)methyl]biphenyl-4-yl}acrylate are obtained in the form of a yellowish oil. Yield=85%

### e. Preparation of methyl 3-{2-butoxy-3′-[((tert-butoxycarbonyl)(methyl)amino)methyl]biphenyl-4-yl}propanoate

In a manner analogous to Example 8h, by reaction of 3.86 g (8.51 mmol, 1 eq.) of methyl (E)-3-{2-butoxy-3′-[((tert-butoxycarbonyl)(methyl)amino)methyl]biphenyl-4-yl}acrylate in 10 ml of methanol in the presence of 386 mg (10% by weight) of 10% palladium-on-charcoal, 3.32 g of methyl 3-{2-butoxy-3′-[((tert-butoxycarbonyl)(methyl)amino)methyl]biphenyl-4-yl}propanoate are obtained in the form of a colorless oil. Yield=86%

### f. Preparation of methyl 3-(2-butoxy-3′-(methylaminomethyl)biphenyl-4-yl)propanoate

A solution of 3.31 g (7.27 mmol, 1 eq.) of methyl 3-{2-butoxy-3′-[((tert-butoxycarbonyl)(methyl)amino)methyl]biphenyl-4-yl}propanoate in 40 ml of dichloromethane in the presence of 5.4 ml (72.7 mmol, 10 eq.) of trifluoroacetic acid is stirred at ambient temperature for 2 hours. The solvents are evaporated and the oil obtained is chromatographed on silica gel (FlashSmart column 120 g) eluted with dichloromethane/methanol (97/3). 3.32 g of methyl 3-(2-butoxy-3′-(methylaminomethyl)biphenyl-4-yl)propanoate are obtained in the form of a whitish oil. Yield=100%

### g. Preparation of methyl 3-{2-butoxy-3′-[((hexanoyl)(methyl)amino)methyl]biphenyl-4-yl}propanoate

A solution of 480 mg (1.35 mmol, 1 eq.) of methyl 3-(2-butoxy-3′-(methylaminomethyl)biphenyl-4-yl)propanoate and of 377 μl (2.70 mmol, 2 eq.) of hexanoyl chloride in 15 ml of dichloromethane in the presence of 560 μl (4.05 mmol, 3 eq.) of triethylamine and of 16 mg (10 mol %) of 4-dimethylaminopyridine is stirred at ambient temperature for 16 hours. The reaction medium is hydrolyzed with a 2N hydrochloric acid solution and separated by settling. The organic phase is washed with water and dried over magnesium sulfate. The solvent is evaporated and the oil obtained is chromatographed on silica gel (FlashSmart column 35 g) eluted with heptane/ethyl acetate (70/30). 400 mg of methyl 3-{2-butoxy-3′-[((hexanoyl)(methyl)amino)methyl]biphenyl-4-yl}propanoate are obtained in the form of an oil. Yield=65%

### h. Synthesis of 3-{2-butoxy-3′-[((hexanoyl)(methyl)amino)methyl]biphenyl-4-yl}propanoic acid

A solution of 390 mg (0.86 mmol, 1 eq.) of methyl 3-{2-butoxy-3′-[((hexanoyl)(methyl)amino)methyl]biphenyl-4-yl}propanoate and of 4.3 ml (4.3 mmol, 5 eq.) of a 1N lithium hydroxide solution in 10 ml of tetrahydrofuran is stirred at ambient temperature for 20 hours. The reaction medium is concentrated, acidified with a 2N hydrochloric acid solution and then extracted with ethyl acetate. The organic phases are combined, washed with water and dried over magnesium sulfate. The solvent is evaporated and the oil obtained is chromatographed on silica gel (FlashSmart column 20 g) eluted with dichloromethane/methanol (98/2). 378 mg of 3-{2-butoxy-3′-[((hexanoyl)(methyl)amino)methyl]biphenyl-4-yl}propanoic acid are obtained in the form of a yellowish oil. Yield=100%

¹H NMR (CDCl₃, 400 MHz): 0.86-0.91 (m, 6H), 1.28-1.41 (m, 6H), 1.65-1.70 (m, 4H), 2.38 (m, 2H), 2.72 (t, 2H), 2.93 (d, 3H), 2.96-3.0 (m, 2H), 3.92-3.96 (m, 2H), 4.56-4.63 (d, 2H), 6.82-6.87 (m, 2H), 7.05-7.15 (d, 1H), 7.22 (t, 1H), 7.33-7.46 (m, 3H).

### EXAMPLE 10

### Synthesis of 3-{2-butoxy-3′-[((methyl)(pentanoyl)amino)methyl]biphenyl-4-yl}propanoic acid

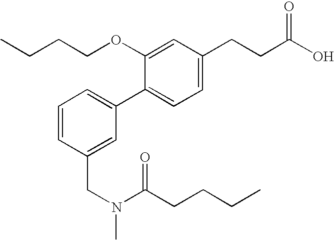

### a. Preparation of methyl 3-{2-butoxy-3′-[((methyl)(pentanoyl)amino)methyl]biphenyl-4-yl}propanoate

In a manner analogous to Example 9g, by reaction of 480 mg (1.35 mmol, 1 eq.) of methyl 3-(2-butoxy-3′-(methylaminomethyl)biphenyl-4-yl)propanoate (prepared in Example 9f) and of 327 μl (2.70 mmol, 2 eq.) of pentanoyl chloride in 15 ml of dichloromethane in the presence of 560 μl (4.05 mmol, 3 eq.) of triethylamine and of 16 mg (10 mol %) of 4-dimethylaminopyridine, 390 mg of methyl 3-{2-butoxy-3′-[((methyl)(pentanoyl)amino)methyl]biphenyl-4-yl}propanoate are obtained in the form of an oil. Yield=66%

### b. Synthesis of 3-{2-butoxy-3′-[((methyl)(pentanoyl)amino)methyl]biphenyl-4-yl}propanoic acid

In a manner analogous to Example 9h, by reaction of 380 mg (0.864 mmol, 1 eq.) of methyl 3-{2-butoxy-3′-[((methyl)(pentanoyl)amino)methyl]biphenyl-4-yl}propanoate and of 4.3 ml (4.3 mmol, 5 eq.) of a 1N lithium hydroxide solution in 10 ml of tetrahydrofuran, 361 mg of 3-{2-butoxy-3′-[((methyl)(pentanoyl)amino)methyl]biphenyl-4-yl}propanoic acid are obtained in the form of a yellowish oil. Yield=98%

¹H NMR (CDCl₃, 400 MHz): 0.87-0.96 (m, 6H), 1.35-1.42 (m, 4H), 1.65-1.70 (m, 4H), 2.38 (m, 2H), 2.73 (t, 2H), 2.94 (d, 3H), 2.98-3.1 (m, 2H), 3.93-3.96 (m, 2H), 4.56-4.63 (d, 2H), 6.82-6.87 (m, 2H), 7.05-7.15 (d, 1H), 7.22 (t, 1H), 7.33-7.44 (m, 3H).

### EXAMPLE 11

### Synthesis of 3-{2-butoxy-3′-[((heptanoyl)(methyl)amino)methyl]biphenyl-4-yl}propanoic acid

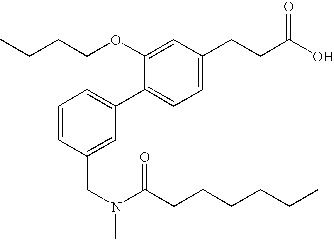

### a. Preparation of methyl 3-{2-butoxy-3′-[((heptanoyl)(methyl)amino)methyl]biphenyl-4-yl}propanoate

In a manner analogous to Example 9g, by reaction of 480 mg (1.35 mmol, 1 eq.) of methyl 3-(2-butoxy-3′-(methylaminomethyl)biphenyl-4-yl)propanoate (prepared in Example 9f) and of 418 μl (2.70 mmol, 2 eq.) of heptanoyl chloride in 15 ml of dichloromethane in the presence of 560 μl (4.05 mmol, 3 eq.) of triethylamine and of 16 mg (10 mol %) of 4-dimethylaminopyridine, 410 mg of methyl 3-{2-butoxy-3′-[((heptanoyl)(methyl)amino)methyl]biphenyl-4-yl}propanoate are obtained in the form of an oil. Yield=65%

### b. Synthesis of 3-{2-butoxy-3′-[((heptanoyl)(methyl)amino)methyl]biphenyl-4-yl}propanoic acid

In a manner analogous to Example 9h, by reaction of 400 mg (0.855 mol, 1 eq.) of methyl 3-{2-butoxy-3′-[((heptanoyl)(methyl)amino)methyl]biphenyl-4-yl}propanoate and of 4.3 ml (4.3 mmol, 5 eq.) of a 1N lithium hydroxide solution in 10 ml of tetrahydrofuran, 378 mg of 3-{2-butoxy-3′-[((heptanoyl)(methyl)amino)methyl]biphenyl-4-yl}propanoic acid are obtained in the form of a yellowish oil. Yield=97.5%

¹H NMR (CDCl₃, 400 MHz): 0.85-0.93 (m, 6H), 1.26-1.41 (m, 8H), 1.67-1.70 (m, 4H), 2.39 (m, 2H), 2.73 (m, 2H), 2.94 (d, 3H), 2.98-3.01 (m, 2H), 3.93-3.96 (m, 2H), 4.56-4.63 (d, 2H), 6.82-6.87 (m, 2H), 7.05-7.15 (d, 1H), 7.22 (t, 1H), 7.33-7.44 (m, 3H).

### EXAMPLE 12

### Synthesis of 3-(2-butoxy-3′-{[(4-ethoxybenzoyl)(methyl)amino]methyl}biphenyl-4-yl)propanoic acid

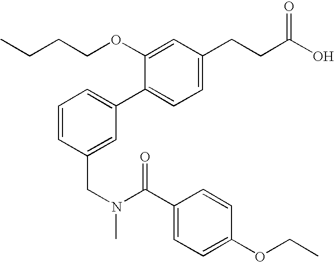

### a. Preparation of methyl 3-(2-butoxy-3′-{[(4-ethoxybenzoyl)(methyl)amino]methyl}biphenyl-4-yl)propanoate

In a manner analogous to Example 9g, by reaction of 400 mg (1.12 mmol, 1 eq.) of methyl 3-(2-butoxy-3′-(methylaminomethyl)biphenyl-4-yl)propanoate (prepared in Example 9f) and of 220 μl (1.40 mmol, 1.2 eq.) of 4-ethoxybenzoyl chloride in 10 ml of dichloromethane in the presence of 500 μl (3.58 mmol, 3.2 eq.) of triethylamine at ambient temperature for 12 hours, methyl 3-(2-butoxy-3′-{[(4-ethoxybenzoyl)(methyl)amino]methyl}biphenyl-4-yl)propanoate is obtained in the form of an oil which is used as is in the following reaction.

### b. Synthesis of 3-(2-butoxy-3′-{[(4-ethoxybenzoyl)(methyl)amino]methyl}biphenyl-4-yl)propanoic acid

In a manner analogous to Example 1e, by reaction of 400 mg (10 mmol) of sodium hydroxide and of the methyl 3-(2-butoxy-3′-{[(4-ethoxybenzoyl)(methyl)amino]methyl}biphenyl-4-yl)propanoate prepared in the preceding stage in 10 ml of tetrahydrofuran/methanol (8/2), 360 mg of 3-(2-butoxy-3′-{[(4-ethoxybenzoyl)(methyl)amino]methyl}biphenyl-4-yl)propanoic acid are obtained in the form of a gum after chromatography on silica gel (heptane/ethyl acetate 70/30). Yield=66% over the 2 stages.

¹H NMR (CDCl₃, 400 MHz): 0.88 (t, 3H), 1.35-1.43 (m & t, 5H), 1.64-1.69 (m, 2H), 2.68 (t, 2H), 2.98 (t & m, 5H), 3.94 (t, 2H), 4.01-4.06 (m, 2H), 4.60 & 4.75 (2m (rotamers), 2H), 6.81 (s, 1H), 6.82-6.87 (m, 3H), 7.10-7.24 (m, 2H), 7.38 (t, 1H), 7.43-7.46 (m, 4H).

### EXAMPLE 13

### Synthesis of 3-(2-butoxy-3′-{[(4-butoxybenzoyl)(methyl)amino]methyl}biphenyl-4-yl)propanoic acid

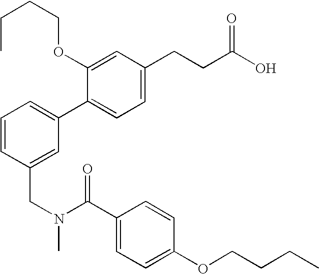

### a. Preparation of methyl 3-(2-butoxy-3′-{[(4-butoxybenzoyl)(methyl)amino]methyl}biphenyl-4-yl)propanoate

In a manner analogous to Example 9g, by reaction of 400 mg (1.12 mmol, 1 eq.) of methyl 3-(2-butoxy-3′-(methylaminomethyl)biphenyl-4-yl)propanoate (prepared in Example 9f) and of 250 μl (1.40 mmol, 1.2 eq.) of 4-butoxybenzoyl chloride in 10 ml of dichloromethane in the presence of 500 μl (3.58 mmol, 3.2 eq.) of triethylamine at ambient temperature for 12 hours, methyl 3-(2-butoxy-3′-{[(4-butoxybenzoyl)(methyl)amino]methyl}biphenyl-4-yl)propanoate is obtained in the form of an oil which is used as is in the following reaction.

### b. Synthesis of 3-(2-butoxy-3′-{[(4-butoxybenzoyl)(methyl)amino]methyl}biphenyl-4-yl)propanoic acid

In a manner analogous to Example 1e, by reaction of 400 mg (10 mmol) of sodium hydroxide and of the methyl 3-(2-butoxy-3′-{[(4-butoxybenzoyl)(methyl)amino]methyl}biphenyl-4-yl)propanoate prepared in the preceding stage in 10 ml of tetrahydrofuran/methanol (8/2), 345 mg of 3-(2-butoxy-3′-{[(4-butoxybenzoyl)(methyl)amino]methyl}biphenyl-4-yl)propanoic acid are obtained in the form of a gum after chromatography on silica gel (heptane/ethyl acetate 70/30). Yield=60% over the 2 stages.

¹H NMR (CDCl₃, 400 MHz): 0.88 (t, 3H), 0.97 (t, 3H), 1.35-1.40 (m, 2H), 1.45-1.51 (m, 2H), 1.64-1.69 (m, 2H), 1.74-1.78 (m, 2H), 2.68 (t, 2H), 2.98 (t & m, 5H), 3.93-3.98 (m, 4H), 4.60 & 4.75 (2m e, 2H), 6.82 (s, H), 6.84-6.88 (m, 3H), 7.10-7.25 (m, 2H), 7.38 (t, 1H), 7.42-7.46 (m, 4H).

### EXAMPLE 14

### Synthesis of 3-{3′-[((benzoyl)(methyl)amino)methyl]-2-butoxybiphenyl-4-yl}propanoic acid

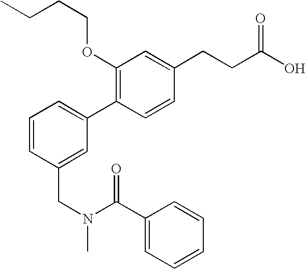

### a. Preparation of methyl 3-{3′-[((benzoyl)(methyl)amino)methyl]-2-butoxybiphenyl-4-yl}propanoate

In a manner analogous to Example 9g, by reaction of 500 mg (1.4 mmol, 1 eq.) of methyl 3-(2-butoxy-3′-(methylaminomethyl)biphenyl-4-yl)propanoate (prepared in Example 9f) and of 325 μl (2.80 mmol, 2 eq.) of benzoyl chloride in 10 ml of dichloromethane in the presence of 600 μl (4.3 mmol, 3 eq.) of triethylamine and of 17 mg (10 mol %) of 4-dimethylaminopyridine, 573 mg of methyl 3-{3′-[((benzoyl)(methyl)amino)methyl]-2-butoxybiphenyl-4-yl}propanoate are obtained in the form of a yellow oil. Yield=89%

### b. Synthesis of 3-{3′-[((benzoyl)(methyl)amino)methyl]-2-butoxybiphenyl-4-yl}propanoic acid

493 mg (12.3 mmol, 10 eq.) of sodium hydroxide are added to a solution of 566 mg (1.23 mmol, 1 eq.) of methyl 3-{3′-[((benzoyl)(methyl)amino)methyl]-2-butoxybiphenyl-4-yl}propanoate in 10 ml of methanol. The reaction mixture is heated at 50° C. for 2 hours. The reaction medium is evaporated to dryness, the residue is taken up in water, acidification is carried out with a 1N hydrochloric acid solution and extraction is carried out with ethyl acetate. The organic phases are combined, washed with water and dried over magnesium sulfate. The solvent is evaporated and the product is crystallized in an ethyl ether/heptane mixture. 450 mg of 3-{3′-[((benzoyl)(methyl)amino)methyl]-2-butoxybiphenyl-4-yl}propanoic acid are obtained in the form of a white powder. (M.p.=103-105° C.) Yield=82%

¹H NMR (CDCl₃, 400 MHz): 0.87 (t, 3H), 1.38 (m, 2H), 1.64-1.70 (m, 2H), 2.73 (t, 2H), 2.88 & 3.06 (2s e, 3H), 2.99 (t, 2H), 3.95 (t, 2H), 4.54 & 4.80 (2s (rotamers), 2H), 6.83 (s, 1H), 6.87 (d, 1H), 7.09-7.51 (m, 10H).

### EXAMPLE 15

### Synthesis of 3-(2-butoxy-3′-{[(4-methoxybenzoyl)(methyl)amino]methyl}biphenyl-4-yl)propanoic acid

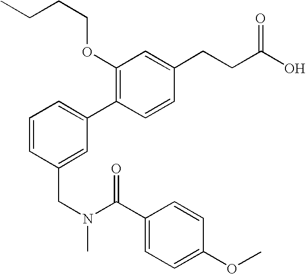

### a. Preparation of methyl 3-(2-butoxy-3′-{[(4-methoxybenzoyl)(methyl)amino]methyl}biphenyl-4-yl)propanoate

In a manner analogous to Example 9g, by reaction of 500 mg (1.4 mmol, 1 eq.) of methyl 3-(2-butoxy-3′-(methylaminomethyl)biphenyl-4-yl)propanoate (prepared in Example 9f) and of 386 μl (2.80 mmol, 2 eq.) of 4-methoxybenzoyl chloride in 10 ml of dichloromethane in the presence of 600 μl (4.3 mmol, 3 eq.) of triethylamine and of 17 mg (10 mol %) of 4-dimethylaminopyridine, 623 mg of methyl 3-(2-butoxy-3′-{[(4-methoxybenzoyl)(methyl)amino]methyl}biphenyl-4-yl)propanoate are obtained in the form of a yellow oil. Yield=91%

### b. Synthesis of 3-(2-butoxy-3′-{[(4-methoxybenzoyl)(methyl)amino]methyl}biphenyl-4-yl)propanoic acid

In a manner analogous to Example 14b, by reaction of 503 mg (12.6 mmol, 10 eq.) of sodium hydroxide and of 616 mg (1.26 mmol, 1 eq.) of methyl 3-(2-butoxy-3′-{[(4-methoxybenzoyl)(methyl)amino]methyl}biphenyl-4-yl)propanoate, prepared in the preceding stage, in 10 ml of methanol, 470 mg of 3-(2-butoxy-3′-{[(4-methoxybenzoyl)(methyl)amino]methyl}biphenyl-4-yl)propanoic acid are obtained in the form of an amorphous solid. (M.p.=39-41° C.) Yield=79%

¹H NMR (CDCl₃, 400 MHz): 0.87 (t, 3H), 1.33-1.42 (m, 2H), 1.64-1.71 (m, 2H), 2.73 (t, 2H), 2.99 (t & m, 5H), 3.81 (s, 3H), 3.95 (t, 2H), 4.60 & 4.75 (2m e, 2H), 6.83-6.89 (m, 4H), 7.23-7.46 (m, 7H).

### EXAMPLE 16

### Synthesis of 3-(2-butoxy-3′-{[(3-methoxybenzoyl)(methyl)amino]methyl}biphenyl-4-yl)propanoic acid

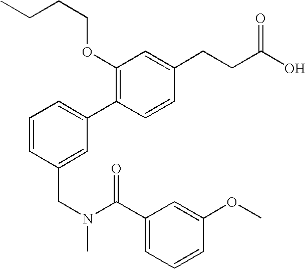

### a. Preparation of methyl 3-(2-butoxy-3′-{[(3-methoxybenzoyl)(methyl)amino]methyl}biphenyl-4-yl)propanoate

In a manner analogous to Example 9g, by reaction of 500 mg (1.4 mmol, 1 eq.) of methyl 3-(2-butoxy-3′-(methylaminomethyl)biphenyl-4-yl)propanoate (prepared in Example 9f) and of 393 μl (2.80 mmol, 2 eq.) of 3-methoxybenzoyl chloride in 10 ml of dichloromethane in the presence of 600 μl (4.3 mmol, 3 eq.) of triethylamine and of 17 mg (10 mol %) of 4-dimethylaminopyridine, 583 mg of methyl 3-(2-butoxy-3′-{[(3-methoxybenzoyl)(methyl)amino]methyl}biphenyl-4-yl)propanoate are obtained in the form of a yellow oil. Yield=85%

### b. Synthesis of 3-(2-butoxy-3′-{[(3-methoxybenzoyl)(methyl)amino]methyl}biphenyl-4-yl)propanoic acid

In a manner analogous to Example 14b, by reaction of 471 mg (11.8 mmol, 10 eq.) of sodium hydroxide and of 576 mg (1.18 mmol, 1 eq.) of methyl 3-(2-butoxy-3′-{[(3-methoxybenzoyl)(methyl)amino]methyl}biphenyl-4-yl)propanoate, prepared in the preceding stage, in 10 ml of methanol, 384 mg of 3-(2-butoxy-3′-{[(3-methoxybenzoyl)(methyl)amino]methyl}biphenyl-4-yl)propanoic acid are obtained in the form of a white powder. (M.p.=80-82° C.) Yield=67%

¹H NMR (CDCl₃, 400 MHz): 0.88 (t, 3H), 1.37 (m, 2H), 1.67 (m, 2H), 2.73 (t, 2H), 2.88 & 3.07 (2s e, 3H), 2.99 (t, 2H), 3.69 & 3.82 (2s (rotamer, 3H), 3.94 (t, 2H), 4.53 & 4.79 (2s (rotamers), 2H), 6.83 (s, 1H), 6.86 (d, 1H), 6.93-7.22 (m, 4H), 7.30-7.50 (m, 5H).

### EXAMPLE 17

### Crossed-Curve PPAR Transactivation Assay

Activation of the PPAR receptors by an agonist (activator) in HeLN cells leads to the expression of a reporter gene, luciferase, which, in the presence of a substrate, generates light. The modulation of the PPAR receptors is measured by quantifying the luminescence produced after incubation of the cells in the presence of a reference agonist. The ligands will displace the agonist from its site. The measurement of the activity is performed by quantifying the light produced. This measurement makes it possible to determine the modulatory activity of the compounds according to the invention by the determination of the constant which is the affinity of the molecule for the PPAR receptor. Since this value can fluctuate depending on the basal activity and the expression of the receptor, it is referred to as apparent Kd (Kdapp in nM).

To determine this constant, “crossed curves” for the test product, against a reference agonist, are prepared using a 96-well plate: 10 concentrations of the test product plus a concentration 0 are arranged in a line, and 7 concentrations of the agonist plus a concentration 0 are arranged in a column. This is 88 measurement points for 1 product and 1 receptor. The remaining 8 wells are used for repeatability controls.

In each well, the cells are in contact with a concentration of the test product and a concentration of the reference agonist, 2-(4-{2-[3-(2,4-difluorophenyl)-1-heptylureido]ethyl}phenylsulfanyl)-2-methylpropionic acid for PPARα, {2-methyl-4-[4-methyl-2-(4-(trifluoromethyl)phenyl)thiazol-5-ylmethylsulfanyl]phenoxy}acetic acid for PPARδ and 5-{4-[2-(methyl(pyrid-2-yl)amino)ethoxy]benzyl}thiazolidine-2,4-dione for PPARγ. Measurements are also taken for total agonist controls with the same products.

The HeLN cell lines used are stable transfectants containing the plasmids ERE-βGlob-Luc-SV-Neo (reporter gene) and PPAR (α, δ, γ) Gal-hPPAR. These cells are seeded in 96-well plates at the rate of 10,000 cells per well in 100 μl of DMEM medium without phenol red and supplemented with 10% of defatted calf serum. The plates are then incubated for 16 hours at 37° C. and 7% CO₂.

The various dilutions of the test products and of the reference ligand are added at the rate of 5 μl per well. The plates are subsequently incubated for 18 hours at 37° C. and 7% CO₂. The culture medium is removed by turning over and 100 μl of a 1:1 PBS/luciferin mixture are added to each well. After 5 minutes, the plates are read using the luminescence reader.

These crossed curves make it possible to determine the AC50 values (concentration at which 50% activation is observed) of the reference ligand at various concentrations of test product. These AC50 values are used to calculate the Schild regression by plotting a straight line corresponding to the Schild equation (“Quantitation in Receptor Pharmacology”, Terry P. Kenakin, Receptors and Channels, 2001, 7, 371-385) which allows the Kdapp values (in nM) to be obtained.

Transactivation Results:

| | PPARα | PPARδ | PPARγ |
|---|---|---|---|
| | Kdapp | Kdapp | Kdapp |
| Compounds | (in nM) | (in nM) | (in nM) |
| Reference 1: 2-(4-{2-[3-(2,4- | 200 | n.a. | n.a. |
| difluorophenyl)-1- | | | |
| heptylureido]ethyl}phenylsulfanyl)- | | | |
| 2-methyl propionic acid | | | |
| Reference 2: {2-methyl-4-[4- | n.a. | 10 | n.a. |
| methyl-2-(4- | | | |
| (trifluoromethyl)phenyl)thiazol-5- | | | |
| ylmethylsulfanyl]phenoxy}acetic | | | |
| acid | | | |
| Reference 3: 5-{4-[2- | n.a. | n.a. | 30 |
| (methyl(pyridin-2- | | | |
| yl)amino)ethoxy]benzyl}thiazolidine- | | | |
| 2,4-dione | | | |
| Example 1 | n.a. | n.a. | 500 |
| Example 2 | n.a. | n.a. | 120 |
| Example 3 | n.a. | n.a. | 60 |
| Example 4 | n.a. | n.a. | 250 |
| Example 6 | n.a. | n.a. | 4000 |
| Example 7 | n.a. | n.a. | 4000 |
| Example 8 | — | — | 1 |
| Example 9 | 4000 | 4000 | 4 |
| Example 10 | 8000 | 4000 | 4 |
| Example 11 | 500 | 4000 | 1 |
| Example 12 | 8000 | 8000 | 0.25 |
| Example 13 | 8000 | 8000 | 1 |
| Example 14 | n.a. | 8000 | 8 |
| Example 15 | n.a. | 4000 | 0.5 |
| Example 16 | n.a. | 4000 | 4 |

n.a. means not active

These results show the affinity of the compounds for PPAR receptors and more particularly the specificity of the affinity of the compounds of the invention for the PPARγ subtype, compared with the affinity of the compounds for the PPARα subtype or for the PPARδ subtype.

### EXAMPLE 18

### Compositions

Various specific formulations based on the compounds according to the invention are illustrated in this example.

A—Oral Route:

(a) 0.2 g tablet:

| | |
|---|---|
| Compound of Example 1 | 0.001 g |
| Starch | 0.114 g |
| Dicalcium phosphate | 0.020 g |
| Silica | 0.020 g |
| Lactose | 0.030 g |
| Talc | 0.010 g |
| Magnesium stearate | 0.005 g |

(b) Suspension to be taken orally in 5 ml vials:

| Compound of Example 5 | 0.001 | g |
|---|---|---|
| Glycerol | 0.500 | g |
| 70% Sorbitol | 0.500 | g |
| Sodium saccharinate | 0.010 | g |
| Methyl para-hydroxybenzoate | 0.040 | g |
| Flavoring | q.s. | |
| Purified water | q.s. for 5 | ml |

(c) 0.2 g tablet:

| | |
|---|---|
| Compound of Example 2 | 0.050 g |
| Lactose monohydrate | 0.132 g |
| Crospovidone | 0.007 g |
| Povidone | 0.005 g |
| Aerosil 200 | 0.004 g |
| Magnesium stearate | 0.002 g |

(d) Suspension to be taken orally in 10 ml vials:

| Compound of Example 4 | 0.200 | g |
|---|---|---|
| Glycerol | 1.000 | g |
| 70% Sorbitol | 1.000 | g |
| Sodium saccharinate | 0.010 | g |
| Methyl para-hydroxybenzoate | 0.080 | g |
| Flavoring | q.s. | |
| Purified water | q.s. for 10 | ml |

B—Topical Route:

(a) Salve:

| | |
|---|---|
| Compound of Example 6 | 0.020 g |
| Isopropyl myristate | 81.700 g |
| Liquid petrolatum | 9.100 g |
| Silica (“Aerosil 200”, marketed by Degussa) | 9.180 g |

(b) Salve:

| | | |
|---|---|---|
| Compound of Example 2 | 0.300 | g |
| White petrolatum, | | |
| pharmaceutical grade | q.s. for 100 | g |

(c) Nonionic water-in-oil cream:

| | | |
|---|---|---|
| Compound of Example 1 | 0.100 | g |
| Mixture of emulsive lanolin alcohols, of waxes | 39.900 | g |
| and of oils (“Anhydrous eucerin”, marketed by BDF) | | |
| Methyl para-hydroxybenzoate | 0.075 | g |
| Propyl para-hydroxybenzoate | 0.075 | g |
| Sterile demineralized water | q.s. for 100 | g |

(d) Lotion:

| | |
|---|---|
| Compound of Example 3 | 0.100 g |
| Polyethylene glycol (PEG) 400 | 69.900 g |
| 95% Ethanol | 30.000 g |

(e) Hydrophobic salve:

| | | |
|---|---|---|
| Compound of Example 5 | 0.300 | g |
| Isopropyl myristate | 36.400 | g |
| Silicone oil (“Rhodorsil 47 V 300”, marketed | 36.400 | g |
| by Rhone-Poulenc) | | |
| Beeswax | 13.600 | g |
| Silicone oil (“Abil 300,000 cSt”, marketed | q.s. for 100 | g |
| by Goldschmidt) | | |

(f) Nonionic oil-in-water cream:

| | | |
|---|---|---|
| Compound of Example 2 | 1.000 | g |
| Cetyl alcohol | 4.000 | g |
| Glyceryl monostearate | 2.500 | g |
| PEG 50 stearate | 2.500 | g |
| Shea butter | 9.200 | g |
| Propylene glycol | 2.000 | g |
| Methyl para-hydroxybenzoate | 0.075 | g |
| Propyl para-hydroxybenzoate | 0.075 | g |
| Sterile demineralized water | q.s. for 100 | g |

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A biaromatic compound having the following general formula (I): 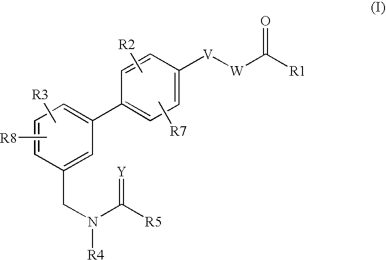
in which:
R1 is a hydroxyl radical, an —OR6 radical or a hydroxylamine radical; wherein R6 is as defined below,
R2 and R3, which may be identical or different, are each a hydrogen atom, a halogen atom, an alkyl radical having from 1 to 12 carbon atoms, a hydroxyl radical, an alkoxy radical, a polyether radical, an aralkyl radical, an aryl radical or an amino radical which can be substituted by one or two identical or different radicals selected from among an alkyl radical having from 1 to 12 carbon atoms or an aralkyl radical;
R4 is a hydrogen atom or a lower alkyl radical having from 1 to 4 carbon atoms;
R5 is an alkyl radical having from 1 to 12 carbon atoms, an aryl radical, an aralkyl radical, a heteroaryl radical, a heterocyclic radical or a 9-fluorenylmethyl radical;
R6 is an alkyl, aryl or aralkyl radical;
R7 and R8, which may be identical or different, are each a hydrogen atom, a halogen atom, an alkyl radical having from 1 to 12 carbon atoms, a hydroxyl radical, an alkoxy radical, a polyether radical, an aralkyl radical, an aryl radical or an amino radical which can be substituted by one or two identical or different radicals selected from among an alkyl radical having from 1 to 12 carbon atoms or an aralkyl radical;
Y is an oxygen or sulfur atom;
V—W is a carbon-carbon single or double bond, namely, a CH₂—CH₂ or CH═CH sequence,
and the salts thereof.

2. The biaromatic compound as defined by claim 1, wherein formula (I), R1 is a hydroxyl radical.

3. The biaromatic compound as defined by claim 1, wherein formula (I), R1 is an —OR6 radical.

4. The biaromatic compound as defined by claim 1, wherein formula (I), R1 is a hydroxylamine radical.

5. The biaromatic compound as defined by claim 1, wherein formula (I), Y is an oxygen atom.

6. The biaromatic compound as defined by claim 1, wherein formula (I), Y is a sulfur atom.

7. The biaromatic compound as defined by claim 1, being a salt of an alkali metal or alkaline earth metal or a salt of an organic amine.

8. The biaromatic compound as defined by claim 1, having an amine functional group, and being a salt of an inorganic acid or a salt of an organic acid.

9. The biaromatic compound as defined by claim 1, having at least one methyl, ethyl, propyl, isopropyl, butyl, tert-butyl, isoamyl, amyl, hexyl, heptyl, octyl, decyl, cyclohexyl, methylcyclohexyl, methylcyclobutyl, methylcyclopentyl or methylcyclohexyl radical substituent.

10. The biaromatic compound as defined by claim 1, having at least one phenyl, biphenyl, cinnamyl or naphthyl radical substituent optionally substituted by a halogen atom, a CF₃ radical, an alkyl radical having from 1 to 12 carbon atoms, an alkoxy radical, a nitro functional group, a polyether radical, an aryl radical, a benzoyl radical, an alkyl ester group, a carboxylic acid, a hydroxyl radical optionally protected by an acetyl or benzoyl group or an amino radical optionally protected by an acetyl or benzoyl group or optionally substituted by at least one alkyl radical having from 1 to 12 carbon atoms, an aralkoxy radical, a phenoxy radical or an amide radical H₂NCO.

11. The biaromatic compound as defined by claim 1, having at least one alkyl radical substituent having from 1 to 12 carbon atoms and substituted by an aryl radical or by a heteroaryl radical.

12. The biaromatic compound as defined by claim 1, having at least one fluorine, chlorine, bromine or iodine atom substituent.

13. The biaromatic compound as defined by claim 1, having at least one methoxy, ethoxy, isopropyloxy, n-propyloxy, tert-butoxy, n-butoxy, n-pentyloxy, n-hexyloxy, cyclopropylmethoxy, cyclobutylmethoxy, cyclopentylmethoxy or cyclohexylmethoxy radical substituent.

14. The biaromatic compound as defined by claim 1, having at least one polyether radical substituent having from 1 to 7 carbon atoms and interrupted by at least one oxygen atom.

15. The biaromatic compound as defined by claim 1, having a pyridyl, furyl, thienyl, isoxazolyl, oxadiazolyl, oxazolyl, isothiazolyl, quinozalinyl, benzothiadiazolyl, benzimidazolyl, indolyl or benzofuranyl radical substituent optionally substituted by at least one halogen, one alkyl radical having from 1 to 12 carbon atoms, one alkoxy radical, one aryl radical, one nitro functional group, one polyether radical, one heteroaryl radical, one benzoyl radical, one alkyl ester group, one carboxylic acid, one hydroxyl optionally protected by an acetyl or benzoyl group or one amino radical optionally protected by an acetyl or benzoyl group or optionally substituted by at least one alkyl radical having from 1 to 12 carbon atoms.

16. The biaromatic compound as defined by claim 1, having a morpholino, pyrrolidino, piperidino, piperazino, 2-oxopiperidin-1-yl or 2-oxopyrrolidin-1-yl radical substituent optionally substituted by at least one alkyl radical having from 1 to 12 carbon atoms, one alkoxy radical, one aryl radical, one nitro functional group, one polyether radical, one heteroaryl radical, one benzoyl radical, one alkyl ester group, one carboxylic acid, one hydroxyl optionally protected by an acetyl or benzoyl group or one amino radical optionally protected by an acetyl or benzoyl group or optionally substituted by at least one alkyl radical having from 1 to 12 carbon atoms.

17. The biaromatic compound as defined by claim 1, selected from the group consisting of:
1. 3-{3′-[((Methyl)(octanoyl)amino)methyl]biphenyl-4-yl}acrylic acid,
2. 3-{3′-[((Methyl)(octanoyl)amino)methyl]biphenyl-4-yl}propanoic acid,
3. 3-{3-Fluoro-3′-[((methyl)(octanoyl)amino)methyl]biphenyl-4-yl}acrylic acid,
4. 3-{3-Fluoro-3′-[((methyl)(octanoyl)amino)methyl]biphenyl-4-yl}propanoic acid,
5. 3-[3′-((Octanoylamino)methyl)biphenyl-4-yl]acrylic acid,
6. 3-[3′-((Octanoylamino)methyl)biphenyl-4-yl]propanoic acid,
7. 3-{3-Hydroxy-3′-[((methyl)(octanoyl)amino)methyl]biphenyl-4-yl}propanoic acid,
8. 3-{2-Butoxy-3′-[((methyl)(octanoyl)amino)methyl]biphenyl-4-yl}propanoic acid,
9. 3-{2-Butoxy-3′-[((hexanoyl)(methyl)amino)methyl]biphenyl-4-yl}propanoic acid,
10. 3-{2-Butoxy-3′-[((methyl)(pentanoyl)amino)methyl]biphenyl-4-yl}propanoic acid,
11. 3-{2-Butoxy-3′-[((heptanoyl)(methyl)amino)methyl]biphenyl-4-yl}propanoic acid,
12. 3-(2-Butoxy-3′-{[(4-ethoxybenzoyl)(methyl)amino]-methyl}biphenyl-4-yl)propanoic acid,
13. 3-(2-Butoxy-3′-{[(4-butoxybenzoyl)(methyl)amino]-methyl}biphenyl-4-yl)propanoic acid,
14. 3-{3′-[((Benzoyl)(methyl)amino)methyl]-2-butoxy-biphenyl-4-yl}propanoic acid,
15. 3-(2-Butoxy-3′-{[(4-methoxybenzoyl)(methyl)amino]-methyl}biphenyl-4-yl)propanoic acid,
16. 3-(2-Butoxy-3′-{[(3-methoxybenzoyl)(methyl)amino]-methyl}biphenyl-4-yl)propanoic acid,
17. (E)-3-{2-Fluoro-3′-[((methyl)(octanoyl)amino)methyl]biphenyl-4-yl}acrylic acid,
18. 3-{2-Fluoro-3′-[((methyl)(octanoyl)amino)methyl]biphenyl-4-yl}propanoic acid,
19. 3-{2-(2-Methoxyethoxy)-3′-[((methyl)(octanoyl)amino)methyl]biphenyl-4-yl}propanoic acid,
20. 3-{2-(3-Methylbutoxy)-3′-[((methyl)(octanoyl)amino)methyl]biphenyl-4-yl}propanoic acid,
21. 3-{2-(3-Chloropropoxy)-3′-[((methyl)(octanoyl)amino)methyl]biphenyl-4-yl}propanoic acid,
22. 3-{2-Methoxy-3′-[((methyl)(octanoyl)amino)methyl]biphenyl-4-yl}propanoic acid,
23. 3-{2-Methyl-3′-[((methyl)(octanoyl)amino)methyl]biphenyl-4-yl}propanoic acid,
24. 3-(3′-{[Methyl(4-phenylbutyryl)amino]methyl}-biphenyl-4-yl)propanoic acid,
25. (E)-3-{2′-Methyl-5′-[((methyl)(octanoyl)amino)methyl]biphenyl-4-yl}acrylic acid,
26. 3-{2′-Methyl-5′-[((methyl)(octanoyl)amino)methyl]biphenyl-4-yl}propanoic acid,
27. (E)-3-{2-Benzyloxy-3′-[((methyl)(octanoyl)amino)methyl]biphenyl-4-yl}acrylic acid,
28. 3-{2-Benzyloxy-3′-[((methyl)(octanoyl)amino)methyl]biphenyl-4-yl}propanoic acid,
29. 3-{3′-[((Methyl)(octanoyl)amino)methyl]-2-propoxy-biphenyl-4-yl}propanoic acid,
30. (E)-3-{3,5-Difluoro-3′-[((methyl)(octanoyl)amino)methyl]biphenyl-4-yl)acrylic acid,
31. 3-{3,5-Difluoro-3′-[((methyl)(octanoyl)amino)methyl]biphenyl-4-yl}propanoic acid,
32. 3-(3′-{[(4-Butoxybenzoyl)(methyl)amino]methyl}-biphenyl-4-yl)propanoic acid,
33. 3-(3′-{[(4-Butoxybenzoyl)(ethyl)amino]methyl}-biphenyl-4-yl)propanoic acid,
34. 3-{2-Cyclopropylmethoxy-3′-[((methyl)(octanoyl)amino)methyl]biphenyl-4-yl}propanoic acid,
35. 3-{2-Ethoxy-3′-[((methyl)(octanoyl)amino)methyl]biphenyl-4-yl}propanoic acid,
36. 3-[3′-[((Methyl)(octanoyl)amino)methyl]-2-(3,3,3-trifluoropropoxy)biphenyl-4-yl]propanoic acid,
37. 3-[3′-[((Methyl)(octanoyl)amino)methyl]-2-(4,4,4-trifluorobutoxy)biphenyl-4-yl]propanoic acid,
38. 3-{2-(3-Hydroxypropoxy)-3′-[((methyl)(octanoyl)amino)methyl]biphenyl-4-yl}propanoic acid,
39. 3-{2-(4-Hydroxybutoxy)-3′-[((methyl)(octanoyl)amino)methyl]biphenyl-4-yl}propanoic acid,
40. 3-{2-(3-Fluorobenzyloxy)-3′-[((methyl)(octanoyl)amino)methyl]biphenyl-4-yl}propanoic acid,
41. 3-{2-(4-Fluorobenzyloxy)-3′-[((methyl)(octanoyl)amino)methyl]biphenyl-4-yl}propanoic acid,
42. 3-{3′-[((Methyl)(octanoyl)amino)methyl]-2-(pentyloxy)biphenyl-4-yl}propanoic acid,
43. 3-{3′-[((Hexanoyl)(methyl)amino)methyl]-2-(pentyloxy)biphenyl-4-yl}propanoic acid,
44. 3-{2-(2-Ethoxyethoxy)-3′-[((hexanoyl)(methyl)amino)methyl]biphenyl-4-yl}propanoic acid,
45. 3-{2-(2-(Diethylamino)ethoxy)-3′-[((methyl)(octanoyl)amino)methyl]biphenyl-4-yl}propanoic acid,
46. 3-[3′-[((Methyl)(octanoyl)amino)methyl]-2-(2-(morpholin-4-yl)ethoxy)biphenyl-4-yl]propanoic acid,
47. 3-{2-Amino-3′-[((hexanoyl)(methyl)amino)methyl]biphenyl-4-yl}propanoic acid,
48. 3-{2-Butylamino-3′-[((hexanoyl)(methyl)amino)methyl]biphenyl-4-yl}propanoic acid,
49. 3-{2-Benzylamino-3′-[((hexanoyl)(methyl)amino)methyl]biphenyl-4-yl}propanoic acid,
50. 3-{2-Diethylamino-3′-[((hexanoyl)(methyl)amino)methyl]biphenyl-4-yl}propanoic acid,
51. 3-{3′-[((Hexanoyl)(methyl)amino)methyl]-2-(propylamino)biphenyl-4-yl}propanoic acid,
52. 3-{2-(4-Fluorobenzylamino)-3′-[((hexanoyl)(methyl)amino)methyl]biphenyl-4-yl}propanoic acid,
53. 3-{2-Butylamino-3′-[((methyl)(octanoyl)amino)methyl]biphenyl-4-yl}propanoic acid,
54. 3-{2-Benzylamino-3′-[((methyl)(octanoyl)amino)methyl]biphenyl-4-yl}propanoic acid,
55. 3-{2-Diethylamino-3′-[((methyl)(octanoyl)amino)methyl]biphenyl-4-yl}propanoic acid,
56. 3-{3′-[((Methyl)(octanoyl)amino)methyl]-2-(propylamino)biphenyl-4-yl}propanoic acid,
57. 3-{2-(4-Fluorobenzylamino)-3′-[((methyl)(octanoyl)amino)methyl]biphenyl-4-yl}propanoic acid,
58. 3-{2-Cyclohexylmethoxy-3′-[((methyl)(octanoyl)amino)methyl]biphenyl-4-yl}propanoic acid,
59. 3-{2-Cyclopentylmethoxy-3′-[((methyl)(octanoyl)amino)methyl]biphenyl-4-yl}propanoic acid,
60. N-[2′-(2-Cyclobutylethoxy)-4′-(2-(hydroxycarbamoyl)ethyl)biphenyl-3-ylmethyl](methyl)octanamide,
61. 3-[3′-[((Methyl)(octanoyl)amino)methyl]-2-(3-(trifluoromethyl)benzyloxy)biphenyl-4-yl]propanoic acid,
62. 3-[3′-[((Hexanoyl)(methyl)amino)methyl]-2-(4-(trifluoromethyl)benzyloxy)biphenyl-4-yl]propanoic acid,
63. 3-{2-(3-Carbamoylbenzyloxy)-3′-[((methyl)(octanoyl)amino)methyl]biphenyl-4-yl}propanoic acid,
64. 3-[3′-[((Methyl)(octanoyl)amino)methyl]-2-(2-(piperazin-1-yl)ethoxy)biphenyl-4-yl]propanoic acid,
65. 3-[3′-[((Methyl)(octanoyl)amino)methyl]-2-(2-(pyrrolidin-1-yl)ethoxy)biphenyl-4-yl]propanoic acid,
66. 3-[2-(3-Methoxybenzyloxy)-3′-({[3-(3-methoxyphenyl)propionyl](methyl)amino}methyl)biphenyl-4-yl]propanoic acid,
67. 3-[2-(4-(tert-Butyl)benzyloxy)-3′-((methyl[3-(3-phenoxyphenyl)propionyl]amino}methyl)biphenyl-4-yl]propanoic acid,
68. 3-(2-(3,5-Dimethoxybenzyloxy)-3′-{[methyl(3-phenoxybenzoyl)amino]methyl}biphenyl-4-yl)propanoic acid,
69. 3-[3′-{[Methyl(4-phenoxybenzoyl)amino]methyl}-2-(3-(trifluoromethyl)benzyloxy)biphenyl-4-yl]propanoic acid,
70. 3-[2-(3-Isopropoxybenzyloxy)-3′-({[3-(4-methoxyphenyl)propionyl](methyl)amino}methyl)biphenyl-4-yl]propanoic acid,
71. 3-[2′-(3-Methoxybenzyloxy)-5′-({[3-(3-methoxyphenyl)propionyl](methyl)amino}methyl)biphenyl-4-yl]propanoic acid,
72. 3-{2′-Cyclohexylmethoxy-5′-[((hexanoyl)(methyl)amino)methyl]biphenyl-4-yl}propanoic acid,
73. 3-{4′-Ethoxy-3′-[((hexanoyl)(methyl)amino)methyl]-2-propoxybiphenyl-4-yl}propanoic acid,
74. 3-{3′-[((Hexanoyl)(methyl)amino)methyl]-3,5-dimethoxybiphenyl-4-yl}propanoic acid,
75. 3-[3,5-Diethoxy-3′-({[3-(3-methoxyphenyl)propionyl](methyl)amino}methyl)biphenyl-4-yl]propanoic acid,
76. 3-[3′-({[3-(4-Methoxyphenyl)propionyl](methyl)amino}methyl)-3-propoxybiphenyl-4-yl]propanoic acid,
77. 3-{3-Cyclopropylmethoxy-3′-[((hexanoyl)(methyl)amino)methyl]biphenyl-4-yl}propanoic acid,
78. 3-{3-Ethoxy-4′-fluoro-3′-[((hexanoyl)(methyl)amino)methyl]biphenyl-4-yl}propanoic acid,
79. 3-[3′-[((Hexanoyl)(methyl)amino)methyl]-3-(4,4,4-trifluorobutoxy)biphenyl-4-yl]propanoic acid,
80. 3-{3-Benzyloxy-3′-[((hexanoyl)(methyl)amino)methyl]biphenyl-4-yl}propanoic acid,
81. 3-[3′-({[3-(4-Methoxyphenyl)propionyl](methyl)amino}methyl)-3-(3-(trifluoromethyl)benzyloxy)biphenyl-4-yl]propanoic acid,
82. 3-[3′-({[3-(3-Methoxyphenyl)propionyl](methyl)amino}methyl)-3,5-dipropylbiphenyl-4-yl]propanoic acid,
83. 3-{3-(2,2-Dimethylpropyl)-3′-[((hexanoyl)(methyl)amino)methyl]biphenyl-4-yl}propanoic acid,
84. 3-{3′-[((Hexanoyl)(methyl)amino)methyl]-3,5-dimethylbiphenyl-4-yl}propanoic acid,
85. 3-{3-[((Hexanoyl)(methyl)amino)methyl]-4″-methoxy-1,1′;3′,1″-terphenyl-4′-yl}propanoic acid,
86. 3-{3-[((Hexanoyl)(methyl)amino)methyl]-3″-methoxy-1,1′;2′,1″-terphenyl-4′-yl}propanoic acid,
87. 3-[3-({[3-(3-Methoxyphenyl)propionyl](methyl)amino}methyl)-3″-trifluoromethyl-1,1′;2′,1″-terphenyl-4′-yl]propanoic acid,
88. 3-{3′-[((Hexanoyl)(methyl)amino)methyl]-2-[2-(3-isopropoxyphenyl)ethyl]biphenyl-4-yl}propanoic acid,
89. 3-{3′-[((Hexanoyl)(methyl)amino)methyl]-2-[(pyridin-3-ylmethyl)amino]biphenyl-4-yl}propanoic acid,
90. 3-[3′-[((Hexanoyl)(methyl)amino)methyl]-3-(2-methoxyethylamino)biphenyl-4-yl]propanoic acid,
91. Methyl 3-{3,5-diethyl-3′-[((hexanoyl)(methyl)amino)methyl]biphenyl-4-yl}propanoate,
92. Methyl 3-[3′-{[methyl(3-phenoxybenzoyl)amino]-methyl}-2-(3-(trifluoromethyl)benzyloxy)biphenyl-4-yl]propanoate,
93. Methyl 3-[3′-{[(3-(biphenyl-4-yl)propionyl)(methyl)amino]methyl}-2-(3-methoxybenzyloxy)biphenyl-4-yl]propanoate,
94. Ethyl 3-[3′-({[3-(3-methoxyphenyl)propionyl](methyl)amino}methyl)-2-(4,4,4-trifluorobutoxy)biphenyl-4-yl]propanoate,
95. N-[4′-(2-(Hydroxycarbamoyl)ethyl)-4″-methoxy-1,1′;3′,1″-terphenyl-3-ylmethyl](methyl)hexanamide, and mixtures thereof.

18. The biaromatic compound as defined by claim 1, wherein formula (I) at least one of the following conditions is satisfied:
R1 is a hydroxyl radical;
R2 and R7 are each an alkoxy or aryloxy radical, an alkylamino radical or a polyether radical;
R3 and R8 are each a hydrogen atom;
R4 is a lower alkyl radical having from 1 to 4 carbon atoms;
R5 is an alkyl radical having from 3 to 8 carbon atoms or an aryl radical;
Y is an oxygen atom;
the V—W bond is a C—C single or double bond.

19. The biaromatic compound as defined by claim 1, wherein formula (I) all of the following conditions are satisfied:
R1 is a hydroxyl radical;
R2 and R7 are each an alkoxy or aryloxy radical, an alkylamino radical or a polyether radical;
R3 and R8 are each a hydrogen atom;
R4 is a lower alkyl radical having from 1 to 4 carbon atoms;
R5 is an alkyl radical having from 3 to 8 carbon atoms or an aryl radical;
Y is an oxygen atom;
the V—W bond is a C—C single or double bond.

20. A cosmetic composition comprising a cosmetically effective amount of at least one biaromatic compound as defined by claim 1, formulated into a cosmetically and physiologically acceptable vehicle therefor.

21. The cosmetic composition as defined by claim 20, comprising from 0.001% to 3% by weight of said at least one biaromatic compound.

22. The cosmetic composition as defined by claim 20, formulated for body or hair hygiene.

23. A pharmaceutical composition comprising a pharmaceutically effective amount of at least one biaromatic compound as defined by claim 1, formulated into a pharmaceutically and physiologically acceptable vehicle therefor.

24. The pharmaceutical composition as defined by claim 23, comprising from 0.001% to 10% by weight of said at least one biaromatic compound.

25. A regime or regimen for regulating and/or restoring the metabolism of skin lipids, comprising administering to an individual in need of such treatment, a thus effective amount of the pharmaceutical composition as defined by claim 23.

26. A regime or regimen:
1) for treating dermatological conditions or afflictions linked to a disorder of keratinization involving differentiation and proliferation, for treating acne vulgaris, comedonic or polymorphic acne, acne rosacea, nodulocystic acne, acne conglobata, senile acne or secondary acnes, solar, drug or occupational acne,
2) for treating other types of disorders of keratinization, ichthyoses, ichthyosiform conditions, Darier's disease, palmoplantar keratoderma, leucoplakia and leucoplakiform conditions, or cutaneous or mucosal (oral) lichen,
3) for treating other dermatological conditions, disorders or afflictions having an inflammatory immunoallergic component, with or without cell proliferation disorder, and all forms of psoriasis, whether cutaneous, mucosal or ungual, and psoriatic rheumatism, or, alternatively, cutaneous atopy, eczema, or respiratory atopy or, alternatively, gingival hypertrophy,
4) for treating all dermal or epidermal proliferations, whether benign or malignant and whether or not of viral origin, common warts, flat warts and epidermodysplasia verruciformis, florid or oral papillomatoses, T lymphoma, and the proliferations which can be induced by ultraviolet radiation, basal cell and prickle cell epithelioma, and also all precancerous skin lesions, keratoacanthomas,
5) for treating other dermatological disorders, conditions or afflictions, immune dermatoses, lupus erythematosus, immune bullous diseases and collagen diseases, scleroderma,
6) for the treatment of dermatological or general conditions having an immunological component,
7) for the treatment of skin disorders due to exposure to UV radiation, and for repairing or combating skin aging, whether photoinduced or chronologic, or for reducing actinic keratoses and pigmentations, or any pathology associated with chronologic or actinic aging, xerosis,
8) for combating disorders of the sebaceous function, hyperseborrhoea of acne or simple seborrhoea,
9) for preventing or treating disorders of cicatrization or for preventing or repairing stretch marks,
10) for the treatment of disorders of pigmentation, hyperpigmentation, melasma, hypopigmentation or vitiligo,
11) for the treatment of conditions of the metabolism of lipids, obesity, hyperlipidaemia or non-insulin-dependent diabetes,
12) for the treatment of inflammatory conditions, arthritis,
13) for the treatment or prevention of cancerous or precancerous conditions,
14) for the prevention or treatment of alopecia of various origins, alopecia due to chemotherapy or to radiation,
15) for the treatment of disorders of the immune system, asthma, type I diabetes mellitus, multiple sclerosis or other selective dysfunctions of the immune system,
16) for the treatment of conditions of the cardiovascular system, arteriosclerosis or hypertension, comprising administering to an individual in need of such treatment, a thus effective amount of the pharmaceutical composition as defined by claim 23.

27. The biaromatic compound as defined by claim 1, in solid form.
